(19) United States
(12) Patent Application Publication
(10) Pub. No.: US 2005/0163841 A1
(43) Pub. Date: Jul. 28, 2005

(54) Compositions and dosage forms for enhanced absorption of 3-amino-n-butyl-phosphinic acid

(76) Inventors: Patrick S.L. Wong, Burlingame, CA (US); Dong Yan, Cupertino, CA (US); Andrew Lam, So. San Francisco, CA (US); Stephen Hwang, Palo Alto, CA (US); George V. Guittard, Cupertino, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/978,138

(22) Filed: Oct. 29, 2004

Related U.S. Application Data
(60) Provisional application No. 60/519,509, filed on Nov. 12, 2003.
(60) Provisional application No. 60/516,259, filed on Oct. 31, 2003.

(51) Int. Cl. A61K 31/66; A61K 9/22

(52) U.S. Cl. 424/468; 514/114

(57) ABSTRACT

Disclosed are substances, compositions, dosage forms and methods relating to drugs including 3-aminopropyl-n-butyl-phosphinic acid; structural homologs thereof; 3-aminopropyl-n-butyl-phosphinic acid complexes; complexes that comprise structural homologs of 3-aminopropyl-n-butyl-phosphinic acid; pharmaceutically acceptable salts of 3-aminopropyl-n-butyl-phosphinic acid or structural homologs thereof; and mixtures of the above.

### CROSS-REFERENCE TO RELATED APPLICATIONS

This application claims the benefit of U.S. provisional patent application No. 60/519,509, filed Nov. 12, 2003, and of U.S. Provisional Application No. 60/516,259, filed Oct. 31, 2003, both applications are incorporated herein by reference in their entirety.

### FIELD OF THE INVENTION

The invention relates to substances, compositions, dosage forms and methods relating to drugs including 3-aminopropyl-n-butyl-phosphinic acid; structural homologs thereof, 3-aminopropyl-n-butyl-phosphinic acid complexes; complexes that comprise structural homologs of 3-aminopropyl-n-butyl-phosphinic acid; pharmaceutically acceptable salts of 3-aminopropyl-n-butyl-phosphinic acid or structural homologs thereof, and mixtures of the above.

### BACKGROUND

Gamma-aminopropylbutyric acid (GABA) is an important inhibitory neurotransmitter. GABA may be responsible for mediating at least 40% of all inhibitory synaptic events within higher brain centers. GABA receptors include GABA A and GABA B sub-types. Presynaptic GABA B receptors reduce release of various neurotransmitters and neuropeptides. Post-synaptic GABA B receptors are coupled to K+ channels and mediate slow inhibitory postsynaptic potential (IPSP).

GABA B antagonists increase neurotransmitter and neuropeptide release, reduce later IPSP, and increase neuronal excitability. Based on this data, and confirmatory animal experiments, GABA B antagonists are believed to have activity in a variety of CNS indications. These indications include cognition.

3-aminopropyl-n-butyl-phosphinic acid (FIG. 1) was identified as being an orally active GABA B antagonist. The structure, synthesis, and various methods of administration have been disclosed in U.S. Pat. Nos. 5,300,679; 5,190,933; 5,064,819; 5,051,524; 5,013,863; and EP patent 0 319 482. These documents, and all documents cited to herein, are incorporated by reference as if reproduced fully herein.

This compound has been the subject of pharmacokinetic and safety/efficacy studies. In particular, C. H. Gleitner et al., “Human Pharmacokinetics of CGP 36 742, an Orally Active GABA B Antagonist,” Archives of Pharmacology Supplement to Volume 351 (1995) Abstracts of the 36^{th} Spring Meeting 14-17 Mar. 1995, Mainz R12 Abs. 48 (1995); H. J. Mobius et al., “CGP 36 742, an Orally Active GABA B Antagonist: Gender-Related Pharmacokinetics in Healthy Elderly Volunteers,” Abstracts of the 1^{st} Congress of the European Association for Clinical Pharmacology and Therapeutics 27-30 Sep. 1995 Abs 30, Journal de pharmacologie clinique et de therapeutique, vol. 50 suppl. (1995); C. H. Gleitner et al., “Pharmacokinetics of CGP 36 742, an Orally Active GABA B Antagonist, in Humans,” J Clin Pham 36: 428-438 (1996) (“Gleitner 1”); and J. Tomlinson et al., “SGS742, a Novel GABA B Receptor Antagonist, Improves Cognition in Patients with Mild Cognitive Impairment,” 62 Neurology A128 (Suppl 5) (April 2004) (“Tomlinson”) set forth information about 3-aminopropyl-n-butyl-phosphinic acid.

In Tomlinson is disclosed the results of a trial that suggests that patients suffering from Mild Cognitive Impairment (MCI) showed improvements in multiple cognitive domains when treated with 3-aminopropyl-n-butyl-phosphinic acid. However, the disclosed dosing was 600 mg tid. Three times per day dosing (tid) is a problem, especially for medications directed to memory or cognition impaired patients. Dosing multiple times per day can result in non-compliance due to missed doses and confusion about dosing.

Accordingly, a less frequent dosing regimen for this compound would be highly desirable. Very desirably would twice a day (bid) or once per day (qd) dosage forms.

One solution might be to administer the compound less frequently, in larger doses, and in immediate release format. Data found in Gleitner 1 suggests that this strategy may not work. FIG. 4, and accompanying text on page 433, in Gleitner 1 contains data suggesting that neither the dose-corrected area under the curve (AUC) nor the dose-corrected maximum plasma concentration (Cmax) increased for doses above 800 mg.

The articles further stated on page 433 “Dose-corrected AUC decreased with increasing dose . . . in the 800- to 2100-mg dose range. Careful examination of FIG. 4 reveals a dose-corrected AUC value of 40 and 20 μmol·h/(L·mmol) at 600 and 2100 mg dose respectively. Thus, the data indicate a tripling of the dose led to less than doubling of the AUC value. The Gleitner 1 authors concluded, at page 436, that this data suggested a saturable, active mechanism of intestinal absorption. In such a situation, increasing the dose administered would result in an increased GI burden of the drug without matched increase in absorption. This would be an ineffective strategy for reducing dosing frequency while maintaining efficacy.

Accordingly, compositions, dosage forms, and methods are needed to address the problems noted above.

### SUMMARY OF THE INVENTION

The invention relates to a dosage form comprising: (i) a controlled delivery dosing structure comprising structure that controllably delivers a drug; (ii) the drug being selected from the group consisting of 3-aminopropyl-n-butyl-phosphinic acid; structural homologs thereof; complexes that comprise 3-aminopropyl-n-butyl-phosphinic acid or structural homologs thereof; pharmaceutically acceptable salts of 3-aminopropyl-n-butyl-phosphinic acid or structural homologs thereof; and mixtures of the above; wherein at least a portion of the drug is contained by the controlled delivery dosing structure; and
wherein the controlled delivery dosing structure is adapted to controllably deliver the portion of the drug contained by the controlled delivery dosing structure at a rate that is effective to, after a single administration of the dosage form to a patient:
   a. provide a Cmax ranging from about 0.01 to about 700 μmol/L,
   b. provide an AUC from about 30 to about 1500 h·μmol/L, and
   c. maintain a plasma drug concentration that is at least about fifteen percent of the Cmax throughout a window of at least about ten hours duration.

The invention further relates to a method comprising: administering to a patient in need thereof a dosage form comprising a controlled delivery dosing structure comprising structure adapted to controllably deliver a drug, wherein the drug is selected from the group consisting of 3-aminopropyl-n-butyl-phosphinic acid, structural homologs thereof, complexes that comprise 3-aminopropyl-n-butyl-phosphinic acid or structural homologs thereof, pharmaceutically acceptable salts of 3-aminopropyl-n-butyl-phosphinic acid or structural homologs thereof, and mixtures of the above; and wherein at least a portion of the drug is contained by the controlled delivery dosing structure; and controllably delivering the portion of the drug contained by the controlled delivery dosing structure at a rate that is effective to, after a single administration of the dosage form to a patient:
a. provide a Cmax ranging from about 0.01 to about 700 μmol/L,
b. provide an AUC (zero to infinity) from about 30 to about 1500 h·μmol/L, and
c. maintain a plasma drug concentration that is at least about fifteen percent of the Cmax throughout a window of at least about ten hours duration.

The invention also relates to a method comprising: orally delivering a drug to a patient in need thereof at a substantially zero order delivery rate during a window; wherein the drug is selected from the group consisting of 3-aminopropyl-n-butyl-phosphinic acid; structural homologs thereof; complexes that comprise 3-aminopropyl-n-butyl-phosphinic acid or structural homologs thereof; pharmaceutically acceptable salts of 3-aminopropyl-n-butyl-phosphinic acid or structural homologs thereof; and mixtures of the above; and wherein the window has a duration of at least about ten hours.

The invention relates to a dosage form comprising: an oral controlled delivery dosing structure that is adapted to controllably deliver orally a drug at a substantially zero order delivery rate a during a window; wherein the drug is selected from the group consisting of 3-aminopropyl-n-butyl-phosphinic acid; structural homologs thereof; complexes that comprise 3-aminopropyl-n-butyl-phosphinic acid or structural homologs thereof; pharmaceutically acceptable salts of 3-aminopropyl-n-butyl-phosphinic acid or structural homologs thereof; and mixtures of the above; and wherein the window has a duration of at least about ten hours.

The invention relates to a dosage form comprising (i) a controlled delivery dosing structure comprising structure that controllably delivers a drug; (ii) the drug being selected from the group consisting of 3-aminopropyl-n-butyl-phosphinic acid; structural homologs thereof; complexes that comprise 3-aminopropyl-n-butyl-phosphinic acid or structural homologs thereof; pharmaceutically acceptable salts of 3-aminopropyl-n-butyl-phosphinic acid or structural homologs thereof; and mixtures of the above; wherein at least a portion of the drug is contained by the controlled delivery dosing structure; and
wherein the controlled delivery dosing structure is adapted to controllably deliver the portion of the drug contained by the controlled delivery dosing structure in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 24 hrs.

The invention relates to a method of administering to a patient in need thereof a dose of a drug comprising: administering the drug to a patient in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 24 hrs; and wherein the drug is selected from the group consisting of 3-aminopropyl-n-butyl-phosphinic acid; structural homologs thereof; complexes that comprise 3-aminopropyl-n-butyl-phosphinic acid or structural homologs thereof; pharmaceutically acceptable salts of 3-aminopropyl-n-butyl-phosphinic acid or structural homologs thereof; and mixtures of the above.

The invention relates to a substance comprising: a complex that comprises 3-aminopropyl-n-butyl-phosphinic acid or structural homologs thereof; and a transport moiety.

The invention relates to an oral dosage form, comprising a complex that comprises 3-aminopropyl-n-butyl-phosphinic acid or structural homologs thereof, and a C12 alkyl sulfate salt, which complex is present in an amount effective to antagonize gamma-aminopropylbutyric acid B receptors in a patient for a window having a duration of at least about ten hours.

The invention relates to a dosage form comprising: (i) a controlled delivery dosing structure comprising structure that controllably delivers a drug; (ii) the drug comprising a complex that comprises 3-aminopropyl-n-butyl-phosphinic acid or structural homologs thereof, and a C12 alkyl sulfate salt; wherein at least a portion of the drug is contained by the controlled delivery dosing structure; and wherein the controlled delivery dosing structure controllably delivers the portion of the drug contained by the controlled delivery dosing structure at a rate that is effective to, after a single administration of the dosage form to a patient:
a. provide a Cmax ranging from about 0.01 to about 700 μmol/L,
b. provide an AUC (zero to infinity) from about 30 to about 1500 h·μmol/L, and
c. maintain a plasma drug concentration that is at least about fifteen percent of the Cmax throughout a window of at least about ten hours duration.

The invention relates to a method of improving absorption of 3-aminopropyl-n-butyl-phosphinic acid comprising: providing a complex of 3-aminopropyl-n-butyl-phosphinic acid and a transport moiety; and administering the complex to a patient in need thereof.

### BRIEF DESCRIPTION OF THE FIGURES

The following figures are not drawn to scale, and are set forth to illustrate various embodiments of the invention.

FIG. 1 shows the structure of 3-aminopropyl-n-butyl-phosphinic acid.

FIG. 2 is a diagram of epithelial cells of the gastrointestinal tract, illustrating two transport routes of drugs through the epithelium of the G.I. tract.

FIG. 3 shows a diagram of an elementary osmotic pump dosage form.

FIG. 4 shows a diagram of an osmotic dosage form.

FIG. 5 shows a diagram of a tri-layer osmotic dosage form.

FIGS. 6A-6C show diagrams of a controlled release dosage form.

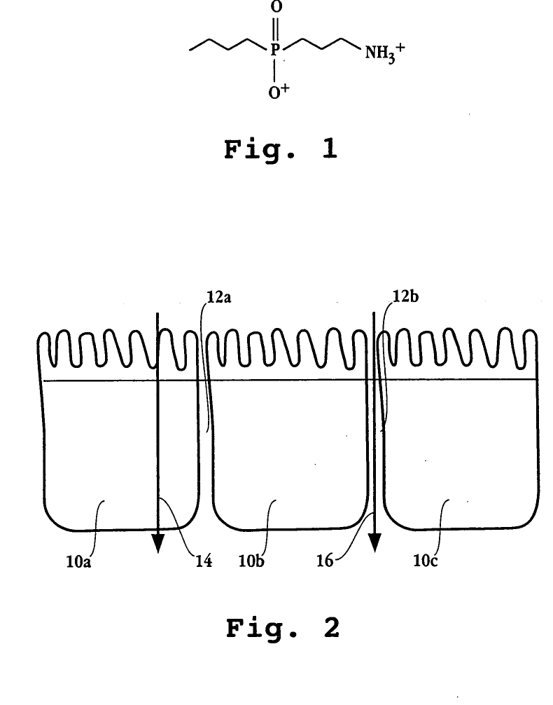

### DETAILED DESCRIPTION

I. Definitions

The present invention is best understood by reference to the following definitions, the drawings and exemplary disclosure provided herein.

By “3ANBPA” is meant a drug or drugs selected from the group consisting of 3-aminopropyl-n-butyl-phosphinic acid; structural homologs thereof; 3-aminopropyl-n-butyl-phosphinic acid complexes; complexes that comprise structural homologs of 3-aminopropyl-n-butyl-phosphinic acid; pharmaceutically acceptable salts of 3-aminopropyl-n-butyl-phosphinic acid or structural homologs thereof; and mixtures of the above.

By “area under the curve” or “AUC” is meant the total area under the plasma drug (3ANBPA) concentration curve. It is calculated from the time of administration to the time point of the last measurable plasma drug concentration using a trapezoidal method plus an extrapolation to infinity according to the ratio of the last measurable plasma drug concentration to the apparent slope of the terminal (natural) log linear portion of the plasma drug concentration profile.

By “C” is meant the concentration of 3ANBPA in blood plasma, or serum, of a subject, generally expressed as mass per unit volume, typically nanograms per milliliter. For convenience, this concentration may be referred to herein as “drug plasma concentration”, “plasma drug concentration” or “plasma concentration” which is intended to be inclusive of 3ANBPA concentration measured in any appropriate body fluid or tissue. The plasma drug concentration at any time following drug administration is referenced as Ctime, as in C9h or C24h, etc.

By “composition” is meant 3ANBPA in combination with additional active pharmaceutical ingredients, and optionally in combination with inactive ingredients, such as pharmaceutically-acceptable carriers, excipients, suspension agents, surfactants, disintegrants, binders, diluents, lubricants, stabilizers, antioxidants, osmotic agents, colorants, plasticizers, and the like.

By “complex” is meant a substance comprising a drug moiety and a transport moiety associated by a tight-ion pair bond. A drug-moiety-transport moiety complex can be distinguished from a loose ion pair of the drug moiety and the transport moiety by a difference in octanol/water partitioning behavior, characterized by the following relationship:
ΔLogD=Log D (complex)−Log D (loose-ion pair)≧0.15 (Equation 1)
wherein:
D, the distribution coefficient (apparent partition coefficient), is the ratio of the equilibrium concentrations of all species of the drug moiety and the transport moiety in octanol to the same species in water (deionized water) at a set pH (typically about pH 5.0 to about pH=7.0) at 25 degrees Celsius. Log D (complex) is determined for a complex of the drug moiety and transport moiety prepared according to the teachings herein. Log D (loose-ion pair) is determined for a physical mixture of the drug moiety and the transport moiety in deionized water. Log D can be determined experimentally or may be predicted for loose-ion pairs using commercially available software packages (e.g., ChemSilico, Inc., Advanced Chemistry Development Inc).

For instance, the octanol/water apparent partition coefficient (D=C_{octanol}/C_{water}) of a putative complex (in deionized water at 25 degree Celsius) can be determined and compared to a 1:1 (mol/mol) physical mixture of the transport moiety and the drug moiety in deionized water at 25 degree Celsius. If the difference between the Log D for the putative complex (D+T−) and the Log D for the 1:1 (mol/mol) physical mixture, D⁺∥T⁻ is determined is greater than or equal to 0.15, the putative complex is confirmed as being a complex according to the invention.

In preferable embodiments, ΔLog D≧0.20, and more preferably ΔLog D≧0.25, more preferably still ΔLog D≧0.35.

By “controlled delivery” or “controllable delivery” is meant continuous or discontinuous release of 3ANBPA over a prolonged period of time, wherein the 3ANBPA is released at (a) a controlled rate over (b) a controlled period of time and in (c) a manner that provides for upper G.I. and lower G.I. tract delivery coupled with improved 3ANBPA absorption as compared to the absorption of 3-aminopropyl-n-butyl-phosphinic acid.

Controlled delivery technologies comprise (i) technologies that improve the lower G.I. tract absorption of 3ANBPA, and (ii) technologies that provide for upper GI tract delivery over a prolonged period of time of 3ANBPA. Technologies that improve the lower G.I. tract absorption of 3ANBPA include, but are not limited to, (i) complexation of forms of 3ANBPA with transport moieties and/or delivery of such complexes to the lower G.I. tract; and (ii) forming prodrugs of forms of 3ANBPA with improved lower G.I. tract absorption and/or delivery of such prodrugs to the lower G.I. tract. In a preferred embodiment, 3ANBPA is controllably delivered by complexation of 3ANBPA with alkyl sulfates coupled with delivery of such complexes to the upper and lower G.I. tract. Technologies that provide for upper GI delivery over a prolonged period of time of 3ANBPA comprise gastric retention systems.

By “dosage form” is meant a pharmaceutical composition in a medium, carrier, vehicle, or device suitable for administration to a patient in need thereof.

By “drug” or “drug moiety” is meant a drug, compound, or agent, or a residue of such a drug, compound, or agent that provides some pharmacological effect when administered to a subject. For use in forming a complex, the drug comprises a(n) acidic, basic, or zwitterionic structural element, or a(n) acidic, basic, or zwitterionic residual structural element. In embodiments according to the invention, drug moieties that comprise acidic structural elements or acidic residual structural elements are complexed with transport moieties that comprise basic structural elements or basic residual structural elements. In embodiments according to the invention, drug moieties that comprise basic structural elements or basic residual structural elements are complexed with transport moieties that comprise acidic structural elements or acidic residual structural elements. In embodiments according to the invention, drug moieties that comprise zwitterionic structural elements or zwitterionic residual structural elements are complexed with transport moieties that comprise either acidic or basic structural elements, or acidic or basic residual structural elements. In an embodiment, the pKa of an acidic structural element or acidic residual structural element is less than about 7.0, preferably less than about 6.0. In an embodiment, the pKa of a basic structural element or basic residual structural element is greater than about 7.0, preferably greater than about 8.0. Zwitterionic structural elements or zwitterionic residual structural elements are analyzed in terms of their individual basic structural element or basic residual structural element or their acidic structural element or acidic residual structural element, depending upon how the complex with the transport moiety is to be formed.

By “fatty acid” is meant any of the group of organic acids of the general formula CH₃(CₙHₓ)COOH where the hydrocarbon chain is either saturated (x=2n, e.g. palmitic acid, CH₃C₁₄H₂₈COOH) or unsaturated (for monounsaturated, x=2n−2, e.g. oleic acid, CH₃C₁₆H₃₀COOH).

By “intestine” or “gastrointestinal (G.I.) tract” is meant the portion of the digestive tract that extends from the lower opening of the stomach to the anus, composed of the small intestine (duodenum, jejunum, and ileum) and the large intestine (ascending colon, transverse colon, descending colon, sigmoid colon, and rectum).

By “loose ion-pair” is meant a pair of ions that, at physiologic pH and in an aqueous environment, are readily interchangeable with other loosely paired or free ions that may be present in the environment of the loose ion pair. Loose ion-pairs can be found experimentally by noting interchange of a member of a loose ion-pair with another ion, at physiologic pH and in an aqueous environment, using isotopic labeling and NMR or mass spectroscopy. Loose ion-pairs also can be found experimentally by noting separation of the ion-pair, at physiologic pH and in an aqueous environment, using reverse phase HPLC. Loose ion-pairs may also be referred to as “physical mixtures,” and are formed by physically mixing the ion-pair together in a medium.

By “lower gastrointestinal tract” or “lower G.I. tract” is meant the large intestine.

By “patient” is meant an animal, preferably a mammal, more preferably a human, in need of therapeutic intervention.

By “pharmaceutically acceptable salt” is meant any salt of a low solubility and/or low dissolution rate free acid pharmaceutical agent whose cation does not contribute significantly to the toxicity or pharmacological activity of the salt, and, as such, they are the pharmacological equivalents of the low solubility and/or low dissolution rate free acid pharmaceutical agent. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base.

By “pharmaceutical composition” is meant a composition suitable for administration to a patient in need thereof.

By “prolonged period of time” is meant a continuous period of time of greater than about 1 hour, preferably, greater than about 4 hours, more preferably, greater than about 8 hours, more preferably greater than about 10 hours, more preferably still, greater than about 14 hours, most preferably, greater than about 14 hours and up to about 24 hours.

As used herein, unless otherwise noted, “rate of release” or “release rate” of a drug refers to the quantity of drug released from a dosage form per unit time, e.g., milligrams of drug released per hour (mg/hr). Drug release rates for dosage forms are typically measured as an in vitro rate of drug release, i.e., a quantity of drug released from the dosage form per unit time measured under appropriate conditions and in a suitable fluid.

The release rates referred to herein are determined by placing a dosage form to be tested in de-ionized water in metal coil or metal cage sample holders attached to a USP Type VII bath indexer in a constant temperature water bath at 37° C. Aliquots of the release rate solutions, collected at pre-set intervals, are then injected into a chromatographic system fitted with an ultraviolet or refractive index detector to quantify the amounts of drug released during the testing intervals.

As used herein a drug release rate obtained at a specified time refers to the in vitro release rate obtained at the specified time following implementation of the release rate test. The time at which a specified percentage of the drug within a dosage form has been released from said dosage form is referred to as the “Tx” value, where “x” is the percent of drug that has been released. For example, a commonly used reference measurement for evaluating drug release from dosage forms is the time at which 70% of drug within the dosage form has been released. This measurement is referred to as the “T70” for the dosage form. Preferably, T70 is greater than or equal to about 8 hours, more preferably, T70 is greater than or equal to about 12 hours, more preferably still, T70 is greater than to equal to about 16 hours, most preferably, T70 is greater than or equal to about 20 hours. In one embodiment, T70 is greater than or equal to about 12 hours and less than about 24 hours. In another embodiment, T70 is greater than or equal to about 8 hours and less than about 16 hours.

By “residual structural element” is meant a structural element that is modified by interaction or reaction with another compound, chemical group, ion, atom, or the like. For example, a carboxyl structural element (COOH) interacts with sodium to form a sodium-carboxylate salt, the COO− being a residual structural element.

By “solvent(s)” is meant a substance in which various other substances may be fully or partially dissolved. In the present invention, preferred solvents include aqueous solvents, and solvents having a dielectric constant less than that of water. Preferred solvents having a dielectric constant less than that of water. The dielectric constant is a measure of the polarity of a solvent and dielectric constants for exemplary solvents are shown in Table 1.

TABLE 1
CHARACTERISTICS OF EXEMPLARY SOLVENTS

| Solvent | Boiling Pt., ° C. | Dielectric constant |
|---|---|---|
| Water | 100 | 80 |
| Methanol | 68 | 33 |
| Ethanol | 78 | 24.3 |
| 1-propanol | 97 | 20.1 |
| 1-butanol | 118 | 17.8 |
| acetic acid | 118 | 6.15 |
| Acetone | 56 | 20.7 |
| methyl ethyl ketone | 80 | 18.5 |
| ethyl acetate | 78 | 6.02 |
| Acetonitrile | 81 | 36.6 |
| N,N-dimethylformamide (DMF) | 153 | 38.3 |
| diemthyl sulfoxide (DMSO) | 189 | 47.2 |
| Hexane | 69 | 2.02 |
| Benzene | 80 | 2.28 |
| diethyl ether | 35 | 4.34 |
| tetrahydrofuran (THF) | 66 | 7.52 |
| methylene chloride | 40 | 9.08 |
| carbon tetrachloride | 76 | 2.24 |

The solvents water, methanol, ethanol, 1-propanol, 1-butanol, and acetic acid are polar protic solvents having a hydrogen atom attached to an electronegative atom, typically oxygen. The solvents acetone, ethyl acetate, methyl ethyl ketone, and acetonitrile are dipolar aprotic solvents, and are in one embodiment, preferred for use in forming the inventive complexes. Dipolar aprotic solvents do not contain an OH bond but typically have a large bond dipole by virtue of a multiple bond between carbon and either oxygen or nitrogen. Most dipolar aprotic solvents contain a C—O double bond. Solvents having a dielectric constant less than that of water are particularly useful in the formation of the inventive complexes. The dipolar aprotic solvents noted in Table 1 have a dielectric constant at least two-fold lower than water and a dipole moment close to or greater than water.

By “structural element” is meant a chemical group that (i) is part of a larger molecule, and (ii) possesses distinguishable chemical functionality. For example, an acidic group or a basic group on a compound is a structural element.

By “structural homolog” is meant compounds with structures chemically similar to 3-aminopropyl-n-butyl-phosphinic acid. Structural homologs comprise prodrugs of 3-aminopropyl-n-butyl-phosphinic acid, and/or derivatives of 3-aminopropyl-n-butyl-phosphinic acid that have been modified to be substrates of active transporters found in the lower G.I. tract.

By “substance” is meant a chemical entity having specific characteristics.

By “tight-ion pair” is meant a pair of ions that are, at physiologic pH and in an aqueous environment are not readily interchangeable with other loosely paired or free ions that may be present in the environment of the tight-ion pair. A tight-ion pair can be experimentally detected by noting the absence of interchange of a member of a tight ion-pair with another ion, at physiologic pH and in an aqueous environment, using isotopic labeling and NMR or mass spectroscopy. Tight ion pairs also can be found experimentally by noting the lack of separation of the ion-pair, at physiologic pH and in an aqueous environment, using reverse phase HPLC.

By “therapeutically effective amount” is meant that amount of 3ANBPA that elicits the biological or medicinal response in a tissue system, animal or human that is being sought by a researcher, veterinarian, medical doctor or other clinician, which includes alleviation of the symptoms of the disease or disorder being treated.

By “transport moiety” is meant a compound that is capable of forming, or a residue of that compound that has formed, a complex with a drug, wherein the transport moiety serves to improve transport of the drug across epithelial tissue, compared to that of the uncomplexed drug. The transport moiety comprises a hydrophobic portion and a(n) acidic, basic, or zwitterionic structural element, or a(n) acidic, basic, or zwitterionic residual structural element. In a preferred embodiment, the hydrophobic portion comprises a hydrocarbon chain. In an embodiment, the pKa of a basic structural element or basic residual structural element is greater than about 7.0, preferably greater than about 8.0. Zwitterionic structural elements or zwitterionic residual structural elements are analyzed in terms of their individual basic structural element or basic residual structural element or their acidic structural element or acidic residual structural element, depending upon how the complex with the drug moiety is to be formed.

In a more preferred embodiment, transport moieties comprise pharmaceutically acceptable acids, including but not limited to carboxylic acids, and salts thereof. In embodiments, transport moieties comprise fatty acids or its salts, benzenesulfonic acid or its salts, benzoic acid or its salts, fumaric acid or its salts, or salicylic acid or its salts. In preferred embodiments the fatty acids or their salts, comprise from 6 to 18 carbon atoms (C6-C18), more preferably 8 to 16 carbon atoms (C8-C16), even more preferably 10 to 14 carbon atoms (C10-C14), and most preferably 12 carbon atoms (C12).

In more preferred embodiments, transport moieties comprise alkyl sulfates (either saturated or unsaturated) and their salts, such as potassium, magnesium, and sodium salts, including particularly sodium octyl sulfate, sodium decyl sulfate, sodium lauryl sulfate, and sodium tetradecyl sulfate. In preferred embodiments the alkyl sulfate or its salt comprise from 6 to 18 carbon atoms (C6-C18), more preferably 8 to 16 carbon atoms (C8-C16), even more preferably 10 to 14 carbon atoms (C10-C14), and most preferably 12 carbon atoms (C12). Also suitable are other anionic surfactants.

In another more preferred embodiment, transport moieties comprise pharmaceutically acceptable primary amines or salts thereof, particularly primary aliphatic amines (both saturated and unsaturated) or salts thereof, diethanolamine, ethylenediamine, procaine, choline, tromethamine, meglumine, magnesium, aluminum, calcium, zinc, alkyltrimethylammonium hydroxides, alkyltrimethylammonium bromides, benzalkonium chloride and benzethonium chloride. Also useful are other pharmaceutically acceptable compounds that comprise secondary or tertiary amines, and their salts, and cationic surfactants.

By “upper gastrointestinal tract” or “upper G.I. tract” is meant that portion of the gastrointestinal tract including the stomach and the small intestine.

By “window” is meant a period of time having a defined duration. Windows preferably begin at time of administration of a dosage form to a patient, or any time thereafter. For instance, in an embodiment a window may have a duration of about 12 hours. In a preferable embodiments, the window may begin at a variety of times. For instance, in a preferable embodiment, the window may begin about 1 hour after administration of a dosage form, and have a duration of about 12 hours, which means that the window would open about 1 hour after administration of the dosage from and close at about 13 hours following administration of the dosage form.

By “zero order rate of release” is meant a rate of release wherein the amount of drug released as a function of time is substantially constant. More particularly, the rate of release of drug as a function of time shall vary by less than about 30%, preferably, less than about 20%, more preferably, less than about 10%, most preferably, less than about 5%, wherein the measurement is taken over the period of time wherein the cumulative release is between about 25% and about 75%, preferably, between about 25% and about 90% by total weight of drug in the dosage form.

### Controlled Delivery of 3-AMINOPROPYL-N-BUTYL-PHOSPHINIC Acid, Complex Formation and Characterization

The inventors have unexpectedly discovered that it is possible to solve the problems in the art discussed above using compositions, dosage forms and methods that deliver 3ANBPA using controlled delivery approaches as set forth herein.

In particular, the inventors note that a conventional solution to the aforementioned problems might be application of conventional controlled release technologies. However, upon close examination of the data, the inventors have discovered that these conventional controlled release technologies are insufficient to solve the aforementioned problems in the art.

The inventors have recognized that the data in Gleitner 1 suggest poor lower G.I. tract absorption. Typically, oral administered dosage forms pass through the entire G.I. tract and are excreted in the feces approximately 24 hours after ingestion. This usually allows for fairly complete absorption of a drug that is not metabolized in the G.I. tract. Gleitner 1, at page 434. reports a cumulative urinary excretion rate of 3-aminopropyl-n-butyl-phosphinic acid of 99.5+/−6.4% of the dose within 24 hours of i.v. administration and 38+/−13% of the dose within 24 hours of oral administration. This implies an absorption through the oral route of only 38% absorption through the oral route. The inventors recognize that this data suggests poor absorption of the drug despite the drug's high solubility.

Further, the Gleitner 1 authors speculate, at page 436, that the drug absorption is facilitated and/or regulated by amino acid transporters. This hypothesis is based in part on the fact that the amount of drug available and Cmax did not increase above single doses of approximately 800 mg, and that drug systemic availability was reduced by 30% after food intake (Gleitner 1, p 436).

The inventors have analyzed this information, and have unexpectedly discovered that 3-aminopropyl-n-butyl-phosphinic acid, in the form reported in the literature, is likely poorly absorbed in the lower G.I. The basis for this discovery is as follows: The data presented in Gleitner 1 are consistent with an active saturable transport mechanism for 3-aminopropyl-n-butyl-phosphinic acid. These types of transporter mechanisms, such as the amino acid transporters suggested in Gleitner 1, are known to be concentrated in the upper G.I. tract. Given that only 38% of the drug is orally absorbed, the inventors have realized that relatively little absorption of the drug must be taking place in the lower G.I. tract.

As a pharmacokinetic understanding, the unexpected recognition that 3-aminopropyl-n-butyl-phosphinic acid is poorly absorbed in the lower G.I. tract accounts for the observations presented in Gleitner 1, and others of the publications cited to herein.

The inventors have further recognized that poor lower G.I. tract absorption implies that neither immediate release (IR) nor conventional controlled release (CR) techniques will work in the development of a bid or qd dosage form of 3-aminopropyl-n-butyl-phosphinic acid. On average, an IR or CR dosage form will move through the upper G.I. tract to the lower G.I. tract within 3-4 hours. Once the dosage form that included 3-aminopropyl-n-butyl-phosphinic acid arrived at the lower G.I. tract, absorption of the compound would be significantly reduced. Therefore, IR or CR dosage forms would have to be dosed more frequently than bid or qd to achieve efficacy. This would be undesirable for 3-aminopropyl-n-butyl-phosphinic acid, as noted above.

Accordingly, the inventors have surprisingly recognized that only a specific sub-class of controlled release technologies, referred to herein as controlled delivery technologies, would suffice to provide bid or qd dosing of 3-aminopropyl-n-butyl-phosphinic acid.

These controlled delivery technologies comprise forms of 3ANBPA that demonstrate improved lower G.I. tract absorption, and technologies that provide for upper G.I. delivery over a prolonged period of time of 3ANBPA. Forms of 3ANBPA that demonstrate improved lower G.I. tract absorption include, but are not limited to, complexation of 3-aminopropyl-n-butyl-phosphinic acid with fatty acids coupled with controlled delivery of the complexes; and forming prodrugs of 3-aminopropyl-n-butyl-phosphinic acid possessing improved lower G.I. absorption coupled with controlled delivery of the prodrugs. In a preferred embodiment, 3ANBPA in the form of a complex is controllably delivered to a patient in need thereof.

Various embodiments of the inventive controlled delivery technologies will now be discussed further herein.

In certain embodiments, 3-aminopropyl-n-butyl-phosphinic acid is modified so as to demonstrate improved lower G.I. tract absorption. Pharmaceutical development typically targets drug forms for absorption in the upper G.I. tract instead of the lower G.I. tract because the upper G.I. tract has a far greater surface area for absorption of drugs than does the lower G.I. tract. The lower G.I. tract lacks microvilli which are present in the upper G.I. tract. The presence of microvilli greatly increases the surface area for drug absorption, and the upper G.I. tract has 480 times the surface area than does the lower G.I. tract. Differences in the cellular characteristics of the upper and lower G.I. tracts also contribute to the poor absorption of molecules in the lower G.I. tract.

FIG. 2 illustrates two common routes for transport of compounds across the epithelium of the G.I. tract. Individual epithelial cells, represented by 10a, 10b, 10c, form a cellular barrier along the small and large intestine. Individual cells are separated by water channels or tight junctions, such as junctions 12a, 12b. Transport across the epithelium occurs via either or both a transcellular pathway and a paracellular pathway. The transcellular pathway for transport, indicated in FIG. 2 by arrow 14, involves movement of the compound across the wall and body of the epithelial cell by passive diffusion or by carrier-mediated transport. The paracellular pathway of transport involves movement of molecules through the tight junctions between individual cells, as indicated by arrow 16. Paracellular transport is less specific but has a much greater overall capacity, in part because it takes place throughout the length of the G.I. tract. However, the tight junctions vary along the length of the G.I. tract, with an increasing proximal to distal gradient in effective ‘tightness’ of the tight junction. Thus, the duodenum in the upper G.I. tract is more “leaky” than the ileum in the upper G.I. tract which is more “leaky” than the colon, in the lower G.I. tract (Knauf, H. et al., Klin. Wochenschr., 60(19): 1191-1200 (1982)).

Since the typical residence time of a drug in the upper G.I. tract is from approximately three to four hours, drugs having poor lower G.I. absorption are absorbed by the body through a period of only three to four hours after oral ingestion. Frequently it is medically desirable that the administered drug be presented in the patient's blood stream at a relatively constant concentration throughout the day. To achieve this with traditional drug formulations that exhibit minimal lower G.I. tract absorption, patients would need to ingest the drugs three to four times a day. Practical experience with this inconvenience to patients suggests that this is not an optimum treatment protocol. The situation with 3-aminopropyl-n-butyl-phosphinic acid is one example.

To provide constant dosing treatments, conventional pharmaceutical development has suggested various controlled release drug systems. Such systems function by releasing their payload of drugs over an extended period of time following administration. However, these conventional forms of controlled release systems are not effective in the case of drugs exhibiting minimal colonic absorption. Since the drugs are only absorbed in the upper G.I. tract and since the residence time of the drug in the upper G.I. tract is only three to four hours, the fact that a proposed controlled release dosage form may release its payload after the residence period of the dosage form in the upper G.I. does not mean the that body will continue to absorb the controlled release drug past the three to four hours of upper G.I. tract residence. Instead, the drug released by the controlled release dosage form after the dosage form has entered the lower G.I. tract is generally not absorbed and, instead, is expelled from the body.

It has been surprisingly found that many common drug moieties with poor absorption characteristics, once complexed with certain transport moieties, exhibit significantly enhanced absorption, particularly lower G.I. tract absorption although upper GI tract absorption may also be enhanced. It is further surprising that complexes, such as certain forms of 3ANBPA, according to the invention show improved absorption as compared to loose ion-pairs (i.e. a non-complexed form) that comprise the same ions as the inventive complexes.

These unexpected results have been found to apply to many categories of drug moieties, including drug moieties that comprise a basic structural element or a basic residual structural element. The unexpected results of the present invention also apply to drug moieties that comprise a zwitterionic structural element or a zwitterionic residual structural element. An example of such a drug moiety comprises 3-aminopropyl-n-butyl-phosphinic acid. The unexpected results of the present invention also apply to drug moieties that comprise an acidic structural element or an acidic residual structural element.

While not wishing to be bound by specific understanding of mechanisms, the inventors reason as follows:

When loose ion-pairs are placed in a polar solvent environment, it is assumed that polar solvent molecules will insert themselves in the space occupied by the ionic bond, thus driving apart the bound ions. A solvation shell, comprising polar solvent molecules electrostatically bonded to a free ion, may be formed around the free ion. This solvation shell then prevents the free ion from forming anything but a loose ion-pairing ionic bond with another free ion. In a situation wherein there are multiple types of counter ions present in the polar solvent, any given loose ion-pairing may be relatively susceptible to counter-ion competition.

This effect is more pronounced as the polarity, expressed as the dielectric constant of the solvent, increases. Based on Coulomb's law, the force between two ions with charges (q1) and (q2) and separated by a distance(r) in a medium of dielectric constant (e) is:

$$F = - \frac{{q}_{1} {q}_{2}}{4 {πɛ}_{0} ɛ {r}^{2}} \qquad ( Equation 2 )$$

where ε₀ is the constant of permittivity of space. The equation shows the importance of dielectric constant (ε) on the stability of a loose ion-pair in solution. In aqueous solution that has a high dielectric constant (ε=80), the electrostatic attraction force is significantly reduced if water molecules attack the ionic bonding and separate the opposite charged ions.

Therefore, high dielectric constant solvent molecules, once present in the vicinity of the ionic bond, will attack the bond and eventually break it. The unbound ions then are free to move around in the solvent. These properties characterize a loose ion-pair.

Tight ion-pairs are formed differently from loose-ion pairs, and consequently possess different properties from a loose ion-pair. Tight ion-pairs are formed by reducing the number of polar solvent molecules in the bond space between two ions. This allows the ions to move tightly together, and results in a bond that is significantly stronger than a loose ion-pair bond, but is still considered an ionic bond. As disclosed more fully herein, tight ion-pairs are obtained using less polar solvents than water so as to reduce entrapment of polar solvents between the ions.

For additional discussion of loose and tight ion-pairs, see D. Quintanar-Guerrero et al., “Applications of the Ion Pair Concept to Hydrophilic Substances with Special Emphasis on Peptides,” Pharm. Res. 14(2): 119-127 (1997).

The difference between loose and tight ion-pairing also can be observed using chromatographic methods. Using reverse phase chromatography, loose ion-pairs can be readily separated under conditions that will not separate tight ion-pairs.

Bonds according to this invention may also be made stronger by selecting the strength of the cation and anion relative to one another. For instance, in the case where the solvent is water, the cation (base) and anion (acid) can be selected to attract one another more strongly. If a weaker bond is desired, then weaker attraction may be selected.

Portions of biological membranes can be modeled to a first order approximation as lipid bilayers for purposes of understanding molecular transport across such membranes. Transport across the lipid bilayer portions (as opposed to active transporters, etc.) is unfavorable for ions because of unfavorable portioning. Various researchers have proposed that charge neutralization of such ions can enhance cross-membrane transport.

In the “ion-pair” theory, ionic drug moieties are paired with transport moiety counter ions to “bury” the charge and render the resulting ion-pair more liable to move through a lipid bilayer. This approach has generated a fair amount of attention and research, especially with regards to enhancing absorption of orally administered drugs across the intestinal epithelium.

While ion-pairing has generated a lot of attention and research, it has not always generated a lot of success. For instance, ion-pairs of two antiviral compounds were found not to result in increased absorption due to the effects of the ion-pair on trans-cellular transport, but rather to an effect on monolayer integrity. The authors concluded that the formation of ion pairs may not be very efficient as a strategy to enhance transepithelial transport of charged hydrophilic compounds as competition by other ions found in in vivo systems may abolish the beneficial effect of counter-ions. J. Van Gelder et al., “Evaluation of the Potential of Ion Pair Formation to Improve the Oral Absorption of two Potent Antiviral Compounds, AMD3100 and PMPA”, Int. J. of Pharmaceutics 186: 127-136 (1999). Other authors have noted that absorption experiments with ion-pairs have not always pointed at clear-cut mechanisms. D. Quintanar-Guerrero et al., Applications of the Ion Pair Concept to Hydrophilic Substances with Special Emphasis on Peptides, Pharm. Res. 14(2): 119-127 (1997).

The inventors have unexpectedly discovered that a problem with these ion-pair absorption experiments is that they were performed using loose-ion pairs, rather than tight ion-pairs. Indeed, many ion-pair absorption experiments disclosed in the art do not even expressly differentiate between loose ion-pairs and tight ion-pairs. One of skill has to distinguish that loose ion-pairs are disclosed by actually reviewing the disclosed methods of making the ion-pairs and noting that such disclosed methods of making are directed to loose ion-pairs not tight ion-pairs. Loose ion-pairs are relatively susceptible to counter-ion competition, and to solvent-mediated (e.g. water-mediated) cleavage of the ionic bonds that bind loose ion-pairs. Accordingly, when the drug moiety of the ion-pair arrives at an intestinal epithelial cell membrane wall, it may or may not be associated in a loose ion-pair with a transport moiety. The chances of the ion-pair existing near the membrane wall may depend more on the local concentration of the two individual ions than on the ion bond keeping the ions together. Absent the two moieties being bound when they approached an intestinal epithelial cell membrane wall, the rate of absorption of the non-complexed drug moiety might be unaffected by the non-complexed transport moiety. Therefore, loose ion-pairs might have only a limited impact on absorption compared to administration of the drug moiety alone.

In contrast, the inventive complexes, particularly the inventive 3ANBPA complexes, are more stable in the presence of polar solvents such as water. Accordingly, the inventors reasoned that, by forming a complex, the drug moiety and the transport moiety would be more likely to be associated as ion-pairs at the time that the moieties would be near the membrane wall. This association would increase the chances that the charges of the moieties would be buried and render the resulting ion-pair more liable to move through the cell membrane.

In an embodiment, the complex comprises a tight ion-pair bond between the drug moiety and the transport moiety. As discussed herein, tight ion-pair bonds are more stable than loose ion-pair bonds, thus increasing the likelihood that the drug moiety and the transport moiety would be associated as ion-pairs at the time that the moieties would be near the membrane wall. This association would increase the chances that the charges of the moieties would be buried and render the tight ion-pair bound complex more liable to move through the cell membrane.

It should be noted that the inventive complexes may improve absorption relative to the non-complexed drug moiety throughout the G.I. tract, not just the lower G.I. tract, as the complex is intended to improve transcellular transport generally, not just in the lower G.I. tract. For instance, if the drug moiety is a substrate for an active transporter found primarily in the upper G.I., the complex formed from the drug moiety may still be a substrate for that transporter. Accordingly, the total transport may be a sum of the transport flux effected by the transporter plus the improved transcellular transport provided by the present invention. In an embodiment, the inventive complex provides improved absorption in the upper G.I. tract, the lower G.I. tract, and both the upper G.I. tract and the lower G.I. tract.

Complexes according to the invention can be made up of a variety of 3ANBPA and transport moieties. Generally speaking, the drug moiety is selected first, and then the appropriate transport moiety is selected to form the inventive complex. One of skill could consider a number of factors in selecting transport moieties, including but not limited to the toxicity and tolerability of the transport moiety, the polarity of the structural element or structural element residue of the drug moiety, the strength of the structural element or structural element residue of the drug moiety, the strength of the structural element or structural element residue of the transport moiety, possible therapeutic advantages of the transport moiety, and the steric hindrance of the bond between the drug moiety and the transport moiety that is provided by the transport moiety. In preferred embodiments, transport moieties comprise alkyl sulfates (either saturated or unsaturated) and their salts, such as potassium, magnesium, and sodium salts, including particularly sodium octyl sulfate, sodium decyl sulfate, sodium lauryl sulfate, and sodium tetradecyl sulfate. In preferred embodiments the alkyl sulfate or its salt comprises from 6 to 18 carbon atoms (C6-C18), more preferably 8 to 16 carbon atoms (C8-C16), even more preferably 10 to 14 carbon atoms (C10-C14), and most preferably 12 carbon atoms (C12). In other preferred embodiments, the transport moieties comprise fatty acids, or their salts, having from 6 to 18 carbon atoms (C6-C18), more preferably 8 to 16 carbon atoms (C8-C16), even more preferably 10 to 14 carbon atoms (C10-C14), and most preferably 12 carbon atoms (C12). Methods of making the inventive 3ANBPA complexes are disclosed herein, including the appended Examples.

An alternative manner of improving lower G.I. absorption of 3-aminopropyl-n-butyl-phosphinic acid is to produce structural homologs of it that are substrates for active transporters expressed in epithelial cells lining the lumen of the human colon. U.S. Patent Application 20030158254 to Zerangue et al., filed Aug. 21, 2003, entitled “Engineering absorption of therapeutic compounds via colonic transporters” (“Zerangue”), hereby incorporated by reference in its entirety for all purposes, discloses drugs modified to be such substrates, including compounds suitable for use in extended release oral dosage forms, particularly those that release drug over periods of greater than about 2-4 hours following administration.

Zerangue discloses a variety of transporters useful in the practice of this invention, comprising the sodium dependent multi-vitamin transporter (SMVT), and monocarboxylate transporters 1 and 4 (MCT 1 and MCT 4). Zerangue also discloses methods of identifying agents or conjugate moieties that are substrates of a transporter, and agents, conjugates, and conjugate moieties that can be screened. In particular, Zerangue discloses compounds to be screened that are variants of known transporter substrates. Such compounds comprise bile salts or acids, steroids, ecosanoids, or natural toxins or analogs thereof, as described by Smith, Am. J. Physiol. 2230, 974-978 (1987); Smith, Am. J. Physiol. 252, G479-G484 (1993); Boyer, Proc. Natl. Acad. Sci. USA 90, 435-438 (1993); Fricker, Biochem. J. 299, 665-670 (1994); Ficker, Biochem J. 299, 665-670 (1994); Ballatori, Am. J. Physiol. 278. Zerangue further discloses the linkage of agents to conjugate moieties, and several compounds, comprising pivaloxymethyl gabaptentin carbamate, gabapentin acetoxyethyl carbamate, and alpha-aminopropylisobutyryl gabapentin.

Structural homologs of 3-aminopropyl-n-butyl-phosphinic acid that are substrates for active transporters expressed in epithelial cells lining the lumen of the human lower G.I. tract are specifically encompassed by the present invention. Such structural homologs can themselves be pharmacologically active, or upon cleavage of a chemical moiety after uptake from the lower G.I. tract, can be metabolized to form a compound that is pharmacologically active (e.g., a prodrug). The structural homologs of 3-aminopropyl-n-butyl-phosphinic acid can be delivered using the controlled delivery technologies disclosed herein.

Exemplary Dosage Forms and Methods of Use

A variety of dosage forms are suitable for use with 3ANBPA. In embodiments, dosage forms that permit dosing that maintains a plasma drug concentration that is at least about fifteen percent of the Cmax throughout a window of at least about ten hours duration are provided. A dosage form may be configured and formulated according to any design that delivers a desired dose of 3ANBPA. In certain embodiments, the dosage form is orally administrable and is sized and shaped as a conventional tablet or capsule. Orally administrable dosage forms may be manufactured according to one of various different approaches. For example, the dosage form may be manufactured as a diffusion system, such as a reservoir device or matrix device, a dissolution system, such as encapsulated dissolution systems (including, for example, “tiny time pills”, and beads) and matrix dissolution systems, and combination diffusion/dissolution systems and ion-exchange resin systems, as described in Remington's Pharmaceutical Sciences, 18th Ed., pp. 1682-1685 (1990).

One important consideration in the practice of this invention is the physical state of the drug substance to be delivered by the dosage form. In certain embodiments, substances comprising 3ANBPA may be in a paste or liquid state, in which case solid dosage forms may not be suitable for use in the practice of this invention. In such cases, dosage forms capable of delivering substances in a paste or liquid state should be used. For instance, an inventive 3ANBPA complex may be in a paste-like state. In such case, dosage forms capable of delivering substances in a paste or liquid state should be used to deliver the complex. Alternatively, in certain embodiments, a different transport moiety may be used to raise the melting point of the substances, thus making it more likely that the inventive complexes will be present in a solid form.

A specific example of a dosage form suitable for use with the present invention is an osmotic dosage form. Osmotic dosage forms, in general, utilize osmotic pressure to generate a driving force for imbibing fluid into a compartment formed, at least in part, by a semipermeable wall that permits free diffusion of fluid but not drug or osmotic agent(s), if present. An advantage to osmotic systems is that their operation is pH-independent and, thus, continues at the osmotically determined rate throughout an extended time period even as the dosage form transits the gastrointestinal tract and encounters differing microenvironments having significantly different pH values. A review of such dosage forms is found in Santus and Baker, “Osmotic drug delivery: a review of the patent literature,” Journal of Controlled Release, 35: 1-21 (1995). Osmotic dosage forms are also described in detail in the following U.S. Patents, each incorporated in their entirety herein: U.S. Pat. Nos. 3,845,770; 3,916,899; 3,995,631; 4,008,719; 4,111,202; 4,160,020; 4,327,725; 4,519,801; 4,578,075; 4,681,583; 5,019,397; and 5,156,850.

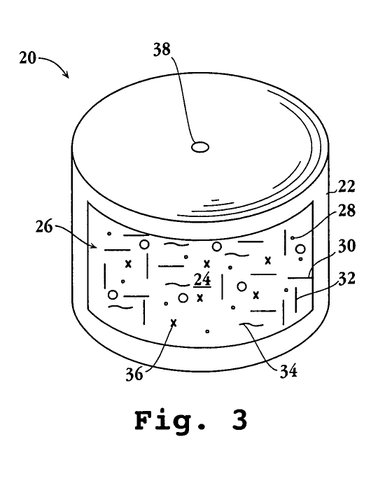

An exemplary dosage form, referred to in the art as an elementary osmotic pump dosage form, is shown in FIG. 3. Dosage form 20, shown in a cutaway view, is also referred to as an elementary osmotic pump, and is comprised of a semi-permeable wall 22 that surrounds and encloses an internal compartment 24. The internal compartment contains a single component layer referred to herein as a drug layer 26, comprising 3ANBPA 28 in an admixture with selected excipients. The excipients are adapted to provide an osmotic activity gradient for attracting fluid from an external environment through wall 22 and for forming a deliverable 3ANBPA formulation upon imbibition of fluid. The excipients may include a suitable suspending agent, also referred to herein as drug carrier 30, a binder 32, a lubricant 34, and an osmotically active agent referred to as an osmagent 36. Exemplary materials for each of these components are provided below.

Semi-permeable wall 22 of the osmotic dosage form is permeable to the passage of an external fluid, such as water and biological fluids, but is substantially impermeable to the passage of components in the internal compartment. Materials useful for forming the wall are essentially nonerodible and are substantially insoluble in biological fluids during the life of the dosage form. Representative polymers for forming the semi-permeable wall include homopolymers and copolymers, such as, cellulose esters, cellulose ethers, and cellulose ester-ethers. Flux-regulating agents can be admixed with the wall-forming material to modulate the fluid permeability of the wall. For example, agents that produce a marked increase in permeability to fluid such as water are often essentially hydrophilic, while those that produce a marked permeability decrease to water are essentially hydrophobic. Exemplary flux regulating agents include polyhydric alcohols, polyalkylene glycols, polyalkylenediols, polyesters of alkylene glycols, and the like.

In operation, the osmotic gradient across wall 22 due to the presence of osmotically-active agents causes gastric fluid to be imbibed through the wall, swelling of the drug layer, and formation of a deliverable 3ANBPA formulation (e.g., a solution, suspension, slurry or other flowable composition) within the internal compartment. The deliverable 3ANBPA formulation is released through an exit 38 as fluid continues to enter the internal compartment. Even as 3ANBPA formulation is released from the dosage form, fluid continues to be drawn into the internal compartment, thereby driving continued release. In this manner, 3ANBPA is released in a sustained and continuous manner over an extended time period.

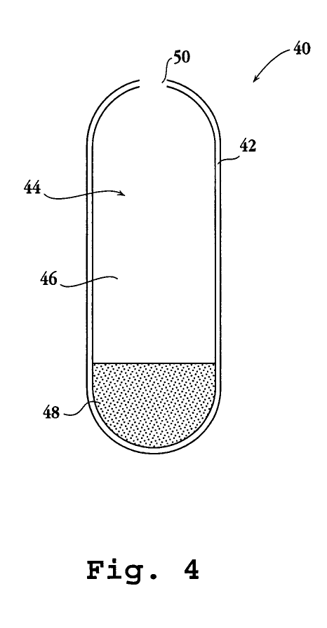

FIG. 4 is a schematic illustration of another exemplary osmotic dosage form. Dosage forms of this type are described in detail in U.S. Pat. Nos. 4,612,008; 5,082,668; and 5,091,190, which are incorporated by reference herein. In brief, dosage form 40, shown in cross-section, has a semi-permeable wall 42 defining an internal compartment 44. Internal compartment 44 contains a bilayered-compressed core having a drug layer 46 and a push layer 48. As will be described below, push layer 48 is a displacement composition that is positioned within the dosage form such that as the push layer expands during use, the materials forming the drug layer are expelled from the dosage form via one or more exit ports, such as exit port 50. The push layer can be positioned in contacting layered arrangement with the drug layer, as illustrated in FIG. 4, or can have one or more intervening layers separating the push layer and drug layer.

Drug layer 46 comprises 3ANBPA in an admixture with selected excipients, such as those discussed above with reference to FIG. 3. An exemplary dosage form can have a drug layer comprised of a complex, a poly(ethylene oxide) as a carrier, sodium chloride as an osmagent, hydroxypropylmethylcellulose as a binder, and magnesium stearate as a lubricant.

Push layer 48 comprises osmotically active component(s), such as one or more polymers that imbibes an aqueous or biological fluid and swells, referred to in the art as an osmopolymer. Osmopolymers are swellable, hydrophilic polymers that interact with water and aqueous biological fluids and swell or expand to a high degree, typically exhibiting a 2-50 fold volume increase. The osmopolymer can be non-crosslinked or crosslinked, and in a preferred embodiment the osmopolymer is at least lightly crosslinked to create a polymer network that is too large and entangled to easily exit the dosage form during use. Examples of polymers that may be used as osmopolymers are provided in the references noted above that describe osmotic dosage forms in detail. A typical osmopolymer is a poly(alkylene oxide), such as poly(ethylene oxide), and a poly(alkali carboxymethylcellulose), where the alkali is sodium, potassium, or lithium. Additional excipients such as a binder, a lubricant, an antioxidant, and a colorant may also be included in the push layer. In use, as fluid is imbibed across the semi-permeable wall, the osmopolymer(s) swell and push against the drug layer to cause release of the drug from the dosage form via the exit port(s).

The push layer can also include a component referred to as a binder, which is typically a cellulose or vinyl polymer, such as poly-n-vinylamide, poly-n-vinylacetamide, poly(vinyl pyrrolidone), poly-n-vinylcaprolactone, poly-n-vinyl-5-methyl-2-pyrrolidone, and the like. The push layer can also include a lubricant, such as sodium stearate or magnesium stearate, and an antioxidant to inhibit the oxidation of ingredients. Representative antioxidants include, but are not limited to, ascorbic acid, ascorbyl palmitate, butylated hydroxyanisole, a mixture of 2 and 3 tertiary-butyl-4-hydroxyanisole, and butylated hydroxytoluene.

An osmagent may also be incorporated into the drug layer and/or the push layer of the osmotic dosage form. Presence of the osmagent establishes an osmotic activity gradient across the semi-permeable wall. Exemplary osmagents include salts, such as sodium chloride, potassium chloride, lithium chloride, etc. and sugars, such as raffinose, sucrose, glucose, lactose, and carbohydrates.

With continuing reference to FIG. 4, the dosage form can optionally include an overcoat (not shown) for color coding the dosage forms according to dose or for providing an immediate release of 3ANBPA or another drug.

In use, water flows across the wall and into the push layer and the drug layer. The push layer imbibes fluid and begins to swell and, consequently, pushes on drug layer 44 causing the material in the layer to be expelled through the exit orifice and into the gastrointestinal tract. Push layer 48 is designed to imbibe fluid and continue swelling, thus continually expelling 3ANBPA from the drug layer throughout the period during which the dosage form is in the gastrointestinal tract. In this way, the dosage form provides a continuous supply of 3ANBPA to the gastrointestinal tract so as to maintain a plasma drug concentration that is at least about fifteen percent of the Cmax throughout a window of at least about ten hours duration. In an alternative embodiment, the dosage form provides a continuous supply of 3ANBPA to the gastrointestinal tract through substantially the entire period of the dosage form's passage through the patient's G.I. tract.

In an embodiment, inventive dosage forms comprise two or more forms of 3ANBPA so that a first form of 3ANBPA is available for absorption in the upper G.I. tract and a second form is presented for absorption in the lower G.I. tract. This can facilitate optimal absorption in circumstances wherein different characteristics are needed to optimize absorption throughout the G.I. tract.

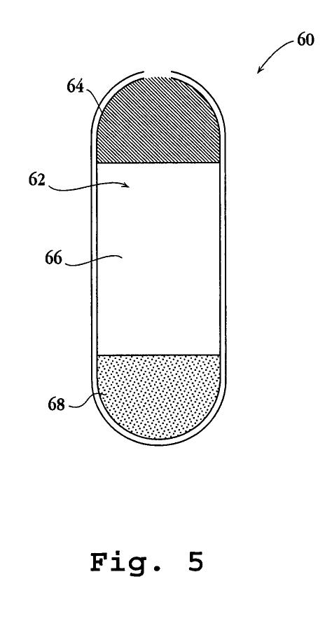

A specific exemplary dosage form comprising a first and second form of 3ANBPA is shown in FIG. 5. Dosage forms of this type are described in detail in U.S. Pat. Nos. 5,545,413; 5,858,407; 6,368,626, and 5,236,689, which are incorporated by reference herein. Osmotic dosage form 60 has a tri-layered core 62 comprised of a first layer 64 of a first form of 3ANBPA, a second layer 66 comprising a second form of 3ANBPA, and a third layer 68 referred to as a push layer. A tri-layered dosage form is prepared to have a first layer of 85.0 wt % of first form of 3ANBPA, 10.0 wt % polyethylene oxide of 100,000 molecular weight, 4.5 wt % polyvinylpyrrolidone having a molecular weight of about 35,000 to 40,000, and 0.5 wt % magnesium stearate. The second layer is comprised 93.0 wt % of a second form of 3ANBPA, 5.0 wt % polyethylene oxide 5,000,000 molecular weight, 1.0 wt % polyvinylpyrrolidone having molecular weight of about 35,000 to 40,000, and 1.0 wt % magnesium stearate.

The push layer consists of 63.67 wt % of polyethylene oxide, 30.00 wt % sodium chloride, 1.00 wt % ferric oxide, 5.00 wt % hydroxypropylmethylcellulose, 0.08 wt % butylated hydroxytoluene and 0.25 wt % magnesium stearate. The semi-permeable wall is comprised of 80.0 wt % cellulose acetate having a 39.8% acetyl content and 20.0% wt polyoxyethylene-polyoxypropylene copolymer.

Dissolution rates of dosage forms, such as those shown in FIGS. 3-5, can be determined according to procedure set forth in Example 3. In general, release of drug formulation from the dosage form begins after contact with an aqueous environment. In the dosage form illustrated in FIG. 3, the drug moiety-transport moiety complex, present in the layer adjacent the exit orifice, is released after contact with an aqueous environment and continues for the lifetime of the device. The dosage form illustrated in FIG. 5 provides an initial release of drug moiety salt, present in the drug layer adjacent the exit orifice, with release of drug moiety-transport moiety complex occurring subsequently. It will be appreciated that this dosage form is designed to release drug moiety salt while in transit in the upper G.I. tract, corresponding approximately to the first eight hours of transit. The complex is released as the dosage form travels through the lower G.I. tract, approximately corresponding to times longer than about 8 hours after ingestion. This design takes advantage of the increased lower G.I. tract absorption provided by the complex.

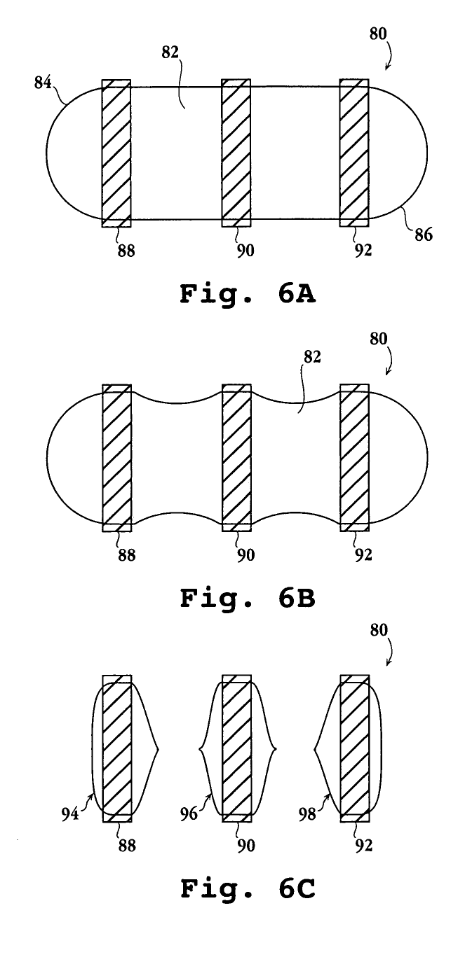

FIGS. 6A-6C illustrate another exemplary dosage form, known in the art and described in U.S. Pat. Nos. 5,534,263; 5,667,804; and 6,020,000, which are specifically incorporated by reference herein. Briefly, a cross-sectional view of a dosage form 80 is shown prior to ingestion into the gastrointestinal tract in FIG. 6A. The dosage form is comprised of a cylindrically shaped matrix 82 comprising 3ANBPA. Ends 84, 86 of matrix 82 are preferably rounded and convex in shape in order to ensure ease of ingestion. Bands 88, 90, and 92 concentrically surround the cylindrical matrix and are formed of a material that is relatively insoluble in an aqueous environment. Suitable materials are set forth in the patents noted above and in Example 6 below.

After ingestion of dosage form 80, regions of matrix 82 between bands 88, 90, 92 begin to erode, as illustrated in FIG. 6B. Erosion of the matrix initiates release of 3ANBPA into the fluidic environment of the G.I. tract. As the dosage form continues transit through the G.I. tract, the matrix continues to erode, as illustrated in FIG. 6C. Here, erosion of the matrix has progressed to such an extent that the dosage form breaks into three pieces, 94, 96, 98. Erosion will continue until the matrix portions of each of the pieces have completely eroded. Bands 94, 96, 98 will thereafter be expelled from the G.I. tract.

In an embodiment, the inventive controlled delivery dosage forms comprise gastric retention dosage forms. U.S. Pat. No. 5,007,790 to Shell, granted Apr. 16, 1991 and entitled Sustained-release oral drug dosage form (“Shell”) discloses a gastric retention dosage form useful in the practice of this invention. Shell discloses sustained-release oral drug-dosage forms that release drug in solution at a rate controlled by the solubility of the drug. The dosage form comprises a tablet or capsule which comprises a plurality of particles of a dispersion of a limited solubility drug in a hydrophilic, water-swellable, crosslinked polymer that maintains its physical integrity over the dosing lifetime but thereafter rapidly dissolves. Once ingested, the particles swell to promote gastric retention and permit the gastric fluid to penetrate the particles, dissolve drug and leach it from the particles. 3ANBPA may be incorporated into such a gastric retention dosage form, or others known in the art (such as disclosed in Example 7), in the practice of this invention.

It will be appreciated the dosage forms described herein are merely exemplary of a variety of dosage forms designed for and capable of achieving delivery of the inventive moiety complex to the G.I. tract. Those of skill in the pharmaceutical arts can identify other dosage forms that would be suitable.

In preferable embodiments, the dosage form comprises a weight equivalent of 3-aminopropyl-n-butyl-phosphinic acid ranging from about 100 mg to about 1500 mg, more preferably ranging from about 300 mg to about 1200 mg, still more preferably ranging from about 400 mg to about 900 mg.

The inventive methods, compositions, dosage forms, and complexes are useful in treating a variety of indications, preferably indications relating to cognitive and memory diseases or disorders.

In an aspect, the invention provides a method for treating an indication, such as a disease or disorder, preferably a cognitive or memory disorder, in a patient by administering a controlled delivery dosage form that comprises 3ANBPA or a composition delivered by controlled delivery that comprises 3ANBPA. In one embodiment, a composition comprising 3ANBPA and a pharmaceutically-acceptable vehicle is administered to the patient via oral administration.

The dose administered is generally adjusted in accord with the age, weight, and condition of the patient, taking into consideration the dosage form and the desired result.

Typical doses of 3ANBPA in the inventive dosage forms may vary broadly. The inventors note that the molecular weight of 3ANBPA may vary significantly depending on whether it is administered as a loose ion-pair salt, a complex, a structural homolog, and so on. Therefore, the dosage strength of 3ANBPA may need to be varied as the form of 3ANBPA incorporated into the dosage form is varied.

As the molecular weight is different for various forms of 3ANBPA, it is confusing to report the dose for a form according to its weight equivalent. It is preferred to report them as the weight equivalent of 3-aminopropyl-n-butyl-phosphinic acid. For instance, the molecular weight of a 3-aminopropyl-n-butyl-phosphinic acid—lauryl sulfate complex is 445.6 mg, and the molecular weight of 3-aminopropyl-n-butyl-phosphinic acid is 179.2. To dose 200 mg weight equivalent of 3-aminopropyl-n-butyl-phosphinic acid, one would need to dose 497.3 mg of 3-aminopropyl-n-butyl-phosphinic acid—lauryl sulfate complex. Using this nomenclature, certain embodiments according to the invention may comprise a weight equivalent of form(s) of 3-aminopropyl-n-butyl-phosphinic acid present in the dosage form ranging from about 100 mg to about 1500 mg, preferably from about 300 mg to about 1200 mg, and more preferably from about 400 mg to about 900 mg. Particular dosage forms may contain about 100 mg, about 200 mg, about 300 mg, about 400 mg, about 450 mg, about 500 mg, about 600 mg, about 700 mg, about 800 mg, about 900 mg, about 1000 mg, about 1200 mg, or about 1500 mg weight equivalents in a given dosage form. Preferred dosing regimens comprise twice daily (e.g. bid) or once per day (e.g. qd) dosing.

The present invention is further directed to a method of treatment comprising administering to a patient in need thereof, an oral controlled delivery dosage form comprising 3ANBPA wherein the 3ANBPA is released from the dosage form at a substantially zero order rate of release, preferably a zero order rate of release. A variety of controlled delivery dosage forms disclosed herein are capable of providing a substantially zero order rate of release, preferably a zero order rate of release. Such dosage forms comprise osmotic dosage forms such as elementary osmotic pumps and other osmotic dosage forms disclosed herein, matrix, as well as others known to one of skill in the art.

The present invention is further directed to pharmaceutical compositions, as that term is defined herein, and to methods of administering pharmaceutical compositions to a patient in need thereof. Preferably the present invention is directed to methods of administering pharmaceutical compositions to a patient in need thereof in therapeutically effective amounts.

In embodiments, the inventive dosage forms may be administered as single or multiple unit dosage forms. In a multiple unit embodiment, for instance, the dosage forms may be administered as two tablets once per day. In a single unit embodiment, for instance, the dosage forms may be administered as one tablet once per day. Other combinations of dosing intervals and single or multiple unit dosage forms are contemplated as being within the scope of the invention.

The invention relates to a dosage form comprising: (i) a controlled delivery dosing structure comprising structure that controllably delivers a drug;
(ii) the drug being selected from the group consisting of 3-aminopropyl-n-butyl-phosphinic acid; structural homologs thereof; complexes that comprise 3-aminopropyl-n-butyl-phosphinic acid or structural homologs thereof; pharmaceutically acceptable salts of 3-aminopropyl-n-butyl-phosphinic acid or structural homologs thereof; and mixtures of the above; wherein at least a portion of the drug is contained by the controlled delivery dosing structure; and wherein the controlled delivery dosing structure is adapted to controllably deliver the portion of the drug contained by the controlled delivery dosing structure at a rate that is effective to, after a single administration of the dosage form to a patient:
   a. provide a Cmax ranging from about 0.01 to about 700 μmol/L,
   b. provide an AUC from about 30 to about 1500 h·μmol/L, and
   c. maintain a plasma drug concentration that is at least about fifteen percent of the Cmax throughout a window of at least about ten hours duration.

Preferably, the invention relates to the dosage form wherein the window has a duration of at least about twelve hours; more preferably wherein the window has a duration of at least about sixteen hours, still more preferably the window has a duration of at least about eighteen hours, yet more preferably window has a duration of at least about twenty hours. In other preferred embodiments, the Cmax ranges from about 10 to about 500 μmol/L; more preferably the Cmax ranges from about 30 to about 300 μmol/L. In other preferred embodiments, the AUC ranges from about 50 to about 1200 h·μmol/L, more preferably the AUC ranges from about 10 to about 1000 h·μmol/L. In other preferred embodiments, the complexes comprise a transport moiety that comprises alkyl sulfate salts, more preferably the transport moiety comprises sodium lauryl sulfate. In other preferred embodiments, the dosage form is a multiple unit dosage form.

The invention relates to a method comprising: administering to a patient in need thereof a dosage form comprising a controlled delivery dosing structure comprising structure adapted to controllably deliver a drug, wherein the drug is selected from the group consisting of 3-aminopropyl-n-butyl-phosphinic acid, structural homologs thereof, complexes that comprise 3-aminopropyl-n-butyl-phosphinic acid or structural homologs thereof, pharmaceutically acceptable salts of 3-aminopropyl-n-butyl-phosphinic acid or structural homologs thereof, and mixtures of the above; and wherein at least a portion of the drug is contained by the controlled delivery dosing structure; and controllably delivering the portion of the drug contained by the controlled delivery dosing structure at a rate that is effective to, after a single administration of the dosage form to a patient:
a. provide a Cmax ranging from about 0.01 to about 700 μmol/L,
b. provide an AUC (zero to infinity) from about 30 to about 1500 h·μmol/L, and
c. maintain a plasma drug concentration that is at least about fifteen percent of the Cmax throughout a window of at least about ten hours duration.

Preferably, the invention relates to the method wherein the window has a duration of at least about twelve hours, more preferably wherein the window has a duration of at least about sixteen hours, still more preferably wherein the window has a duration of at least about eighteen hours, yet more preferably wherein the window has a duration of at least about twenty hours. In other preferable embodiments, the Cmax ranges from about 10 to about 500 μmol/L, more preferably the Cmax ranges from about 30 to about 300 μmol/L. In other preferable embodiments, the AUC ranges from about 50 to about 1200 h·μmol/L, more preferably the AUC ranges from about 10 to about 1000 h·μmol/L. In other preferable embodiments, the complexes comprise a transport moiety that comprises an alkyl sulfate salt, more preferably the transport moiety comprises sodium lauryl sulfate. In other preferable embodiments, the dosage form is a multiple unit dosage form.

The invention relates to a method comprising orally delivering a drug to a patient in need thereof at a substantially zero order delivery rate during a window; wherein the drug is selected from the group consisting of structural homologs of 3-aminopropyl-n-butyl-phosphinic acid; complexes that comprise 3-aminopropyl-n-butyl-phosphinic acid or structural homologs thereof; pharmaceutically acceptable salts of structural homologs of 3-aminopropyl-n-butyl-phosphinic acid; and mixtures of the above; and wherein the window has a duration of at least about ten hours.

Preferably, in the method, the drug is selected from the group consisting of complexes that comprise 3-aminopropyl-n-butyl-phosphinic acid or structural homologs thereof. In other preferable embodiments, the window has a duration of at least about twelve hours, more preferably, the window has a duration of at least about sixteen hours, still more preferably the window has a duration of at least about eighteen hours, yet more preferably the window has a duration of at least about twenty hours.

The invention relates to a dosage form comprising an oral controlled delivery dosing structure that is adapted to controllably deliver orally a drug at a substantially zero order delivery rate a during a window; wherein the drug is selected from the group consisting of structural homologs of 3-aminopropyl-n-butyl-phosphinic acid; complexes that comprise 3-aminopropyl-n-butyl-phosphinic acid or structural homologs thereof; pharmaceutically acceptable salts of structural homologs of 3-aminopropyl-n-butyl-phosphinic acid; and mixtures of the above; and wherein the window has a duration of at least about ten hours.

Preferably, in the dosage form, the drug is selected from the group consisting of complexes that comprise 3-aminopropyl-n-butyl-phosphinic acid or structural homologs thereof. In other preferable embodiments, the window has a duration of at least about twelve hours following oral delivery, more preferably the window has a duration of at least about sixteen hours following oral delivery, still more preferably the window has a duration of at least about eighteen hours following oral delivery, and yet more preferably the window has a duration of at least about twenty hours following oral delivery. In other preferable embodiments, the dosage form is a multiple unit dosage form.

The invention relates to a dosage form comprising (i) a controlled delivery dosing structure comprising structure that controllably delivers a drug; (ii) the drug being selected from the group consisting of structural homologs of 3-aminopropyl-n-butyl-phosphinic acid; complexes that comprise 3-aminopropyl-n-butyl-phosphinic acid or structural homologs thereof; pharmaceutically acceptable salts of structural homologs of 3-aminopropyl-n-butyl-phosphinic acid; and mixtures of the above; wherein at least a portion of the drug is contained by the controlled delivery dosing structure; and
wherein the controlled delivery dosing structure is adapted to controllably deliver the portion of the drug contained by the controlled delivery dosing structure in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 24 hrs.

The invention relates to a method of administering to a patient in need thereof a dose of a drug comprising: administering the drug to a patient in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 24 hrs; and wherein the drug is selected from the group consisting of structural homologs of 3-aminopropyl-n-butyl-phosphinic acid; complexes that comprise 3-aminopropyl-n-butyl-phosphinic acid or structural homologs thereof; pharmaceutically acceptable salts of structural homologs of 3-aminopropyl-n-butyl-phosphinic acid; and mixtures of the above.

The invention relates to a substance comprising: a complex that comprises 3-aminopropyl-n-butyl-phosphinic acid or structural homologs thereof; and a transport moiety. The invention also relates to a composition comprising: the substance and a pharmaceutically acceptable carrier or excipient; in preferable embodiments the pharmaceutically acceptable carrier or excipient comprises an osmagent, a binder, or a lubricant. In preferable embodiments, relating to the substance, the transport moiety comprises an alkyl sulfate salt, more preferably the alkyl sulfate salt comprises a C6-C18 alkyl sulfate salt, still more preferably the C6-C18 alkyl sulfate salt comprises a C1-2 alkyl sulfate salt. The invention also relates to an oral dosage form comprising the composition.

The invention relates to an oral dosage form, comprising a complex that comprises 3-aminopropyl-n-butyl-phosphinic acid or structural homologs thereof, and a C12 alkyl sulfate salt, which complex is present in an amount effective to antagonize gamma-aminopropylbutyric acid B receptors in a patient for a window having a duration of at least about ten hours. Preferably, the window has a duration of at least about 12 hours, more preferably the window has a duration of at least about 16 hours, still more preferably the window has a duration of at least about 20 hours, and yet more preferably the window has a duration of at least about 24 hours. In certain embodiments, the dosage form comprises a weight equivalent of 3-aminopropyl-n-butyl-phosphinic acid ranging from about 100 mg to about 1500 mg, more preferably the dosage form comprises a weight equivalent of 3-aminopropyl-n-butyl phosphinic acid ranging from about 300 mg to about 1200 mg, still more preferably the dosage form comprises a weight equivalent of 3-aminopropyl-n-butyl phosphinic acid ranging from about 400 mg to about 900 mg.

The invention relates to a dosage form comprising: (i) a controlled delivery dosing structure comprising structure that controllably delivers a drug; (ii) the drug comprising a complex that comprises 3-aminopropyl-n-butyl-phosphinic acid or structural homologs thereof, and a C12 alkyl sulfate salt; wherein at least a portion of the drug is contained by the controlled delivery dosing structure; and wherein the controlled delivery dosing structure controllably delivers the portion of the drug contained by the controlled delivery dosing structure at a rate that is effective to, after a single administration of the dosage form to a patient:
a. provide a Cmax ranging from about 0.01 to about 700 μmol/L,
b. provide an AUC (zero to infinity) from about 30 to about 1500 h·μmol/L, and
c. maintain a plasma drug concentration that is at least about fifteen percent of the Cmax throughout a window of at least about ten hours duration.

In preferable embodiments, the window has a duration of at least about twelve hours, more preferably the window has a duration of at least about sixteen hours, still more preferably the window has a duration of at least about eighteen hours, yet more preferably the window has a duration of at least about twenty hours. Preferably, the dosage form comprises a weight equivalent of 3-aminopropyl-n-butyl phosphinic acid ranging from about 100 mg to about 1500 mg, more preferably the dosage form comprises a weight equivalent of 3-aminopropyl-n-butyl phosphinic acid ranging from about 300 mg to about 1200 mg, still more preferably the dosage form comprises a weight equivalent of 3-aminopropyl-n-butyl phosphinic acid ranging from about 400 mg to about 900 mg. In preferable embodiments, the C12 alkyl sulfate salt comprises sodium lauryl sulfate. In preferable embodiments, relating to the dosage form, the Cmax ranges from about 10 to about 500 μmol/L, more preferably the Cmax ranges from about 30 to about 300 μmol/L. In other preferable embodiments, the AUC ranges from about 50 to about 1200 h·μmol/L, more preferably the AUC ranges from about 10 to about 1000 h·μmol/L. In other preferable embodiments, the dosage form is a multiple unit dosage form.

The invention relates to a method of improving absorption of 3-aminopropyl-n-butyl-phosphinic acid comprising: providing a complex of 3-aminopropyl-n-butyl-phosphinic acid and a transport moiety; and administering the complex to a patient in need thereof.

More preferably, the transport moiety comprises an alkyl sulfate salt, still more preferably the alkyl sulfate salt comprises a C6-C18 alkyl sulfate salt, yet more preferably the C6-C18 alkyl sulfate salt comprises a C12 alkyl sulfate salt. In preferable embodiments the complex is administered orally, and the improved absorption comprises improved oral absorption, more preferably the improved oral absorption comprises improved lower gastrointestinal tract absorption or alternatively the improved oral absorption comprises improved upper gastrointestinal tract absorption.

While there has been described and pointed out features and advantages of the invention, as applied to present embodiments, those skilled in the medical art will appreciate that various modifications, changes, additions, and omissions in the method described in the specification can be made without departing from the spirit of the invention.

### EXAMPLES

The following examples are illustrative of the present invention and should not be considered as limiting the scope of the invention in any way, as these examples and other equivalents thereof will become apparent to those versed in the art in light of the present disclosure, drawings and accompanying claims.

### Example 1

### Preparation of 3-aminopropyl-n-butyl-phosphinic Acid—Lauryl Sulfate Complex

The following steps are carried out to form 3-aminopropyl-n-butyl-phosphinic acid—lauryl sulfate complex.
1. 2 mL 5N hydrochloric acid (10 mmol HCl) is dissolved in 50 mL deionized water at room temperature.
2. 10 mmol 3-aminopropyl-n-butyl-phosphinic acid (1.792 g) is added to the solution in step 1. The mixture is stirred to solubilize the 3-aminopropyl-n-butyl-phosphinic acid at room temperature. 3-aminopropyl-n-butyl-phosphinate hydrochloride salt is formed.
3. 10 mmol sodium lauryl sulfate (2.884 g SDS) is added to the solution in step 2. The mixture is stirred to solubilize the SDS at room temperature.
4. 100 mL water insoluble organic solvent is added to the solution in step 3. The mixture is stirred for ˜8 hours at room temperature.
5. The mixture of step 4 is transferred to a separatory funnel. The mixture is then allowed to settle for ˜8 hours. Two phases are formed.
6. The two phases formed in step 5 are separated. Evaporate the organic solution at room temperature to dryness. Further remove the residue of solvent in a vacuum oven for 4 hours at 40° C. Theoretically maximum of 4.456 g product is calculated from the stoicheometric amounts of 3-aminopropyl-n-butyl-phosphinic acid and SDS used.

### Example 2

### In Vivo Absorption Using Flushed Ligated Colonic Model in Rats

An animal model commonly known as the “intracolonic ligated model” is employed for testing formulations. Surgical preparation of a fasted anesthetized 0.3-0.5 kg Sprague-Dawley male rats proceeds as follows. A segment of proximal colon is isolated and the colon is flushed of fecal materials. The segment is ligated at both ends while a catheter is placed in the lumen and exteriorized above the skin for delivery of test formulation. The colonic contents are flushed out and the colon as returned to the abdomen of the animal. Depending on the experimental set up, the test formulation is added after the segment is filled with 1 mL/kg of 20 mM sodium phosphate buffer, pH 7.4, to more accurately simulate the actual colon environment in a clinical situation.

Rats are allowed to equilibrate for approximately 1 hour after surgical preparation and prior to exposure to each test formulation. 3-aminopropyl-n-butyl-phosphinic hydrochloride or a 3-aminopropyl-n-butyl-phosphinic acid-lauryl sulfate complex were administered as an intracolonic bolus at dosages of 10 mg 3-aminopropyl-n-butyl-phosphinic hydrochloride/rat or 10 mg 3-aminopropyl-n-butyl-phosphinic acid-lauryl sulfate complex/rat. Blood samples are obtained from the jugular catheter at 0, 15, 30, 60, 90, 120, 180 and 240 minutes after administration of the test formulation and analyzed for blood 3-aminopropyl-n-butyl-phosphinic acid concentration.

### Example 3

### Preparation of Dosage Form Comprising a 3-Aminopropyl-n-Butyl-Phosphinic Acid—Lauryl Sulfate Complex

A dosage form is prepared as follows:

The 3-aminopropyl-n-butyl-phosphinic acid—lauryl sulfate complex layer in the dosage form is prepared as follows. First, 9.30 grams of 3-aminopropyl-n-butyl-phosphinic acid—lauryl sulfate complex, prepared as described in Example 1, 0.50 g polyethylene oxide of 5,000,000 molecular weight, 0.10 g of polyvinylpyrrolidone having molecular weight of about 38,000 are dry blended in a conventional blender for 20 minutes to yield a homogenous blend. Next, denatured anhydrous ethanol is added slowly to the blend with continuous mixing for 5 minutes. The blended wet composition is passed through a 16 mesh screen and dried overnight at room temperature. Then, the dry granules are passed through a 16 mesh screen and 0.10 g magnesium stearate are added and all the dry ingredients are dry blended for 5 minutes. The composition is comprised of 93.0 wt % 3-aminopropyl-n-butyl-phosphinic acid—lauryl sulfate complex, 5.0 wt % polyethylene oxide 5,000,000 molecular weight, 1.0 wt % polyvinylpyrrolidone having molecular weight of about 35,000 to 40,000 and 1.0 wt % magnesium stearate.

A push layer comprised of an osmopolymer hydrogel composition is prepared as follows. First, 58.67 g of pharmaceutically acceptable polyethylene oxide comprising a 7,000,000 molecular weight, 5 g Carbopol® 974P, 30 g sodium chloride and 1 g ferric oxide are separately screened through a 40 mesh screen. The screened ingredients are mixed with 5 g of hydroxypropylmethylcellulose of 9,200 molecular weight to produce a homogenous blend. Next, 50 mL of denatured anhydrous alcohol is added slowly to the blend with continuous mixing for 5 minutes. Then, 0.080 g of butylated hydroxytoluene is added followed by more blending. The freshly prepared granulation is passed through a 20 mesh screen and allowed to dry for 20 hours at room temperature (ambient). The dried ingredients are passed through a 20 mesh screen and 0.25 g of magnesium stearate was added and all the ingredients are blended for 5 minutes. The final composition is comprised of 58.7 wt % of polyethylene oxide, 30.0 wt % sodium chloride, 5.0 wt % Carbopol® 974P, 5.0 wt % hydroxypropylmethylcellulose, 1.0 wt % ferric oxide, 0.25 wt % magnesium stearate, and 0.08 wt % butylated hydroxytoluene.

The bi-layer dosage form is prepared as follows. First, 535 mg of the 3-aminopropyl-n-butyl-phosphinic acid—lauryl sulfate complex composition is added to a punch and die set and tamped. (497 mg 3-aminopropyl-n-butyl-phosphinic acid—lauryl sulfate complex, or 200 mg 3-aminopropyl-n-butyl-phosphinic acid equivalent) Then, 267 mg of the hydrogel composition is added and the two layers compressed under a compression force of 1.0 ton (1000 kg) into a 9/32 inch (0.714 cm) diameter punch die set, forming an intimate bi-layered core (tablet).

A semipermeable wall-forming composition is prepared comprising 80.0 wt % cellulose acetate having a 39.8% acetyl content and 20.0% polyoxyethylene-polyoxypropylene copolymer having a molecular weight of 7680-9510 by dissolving the ingredients in acetone in a 80:20 wt/wt composition to make a 5.0% solids solution. Placing the solution container in a warm water bath during this step accelerates the dissolution of the components. The wall-forming composition is sprayed onto and around the bi-layered core to provide a 60 to 80 mg thickness semi-permeable wall.

Next, a 40 mil (1.02 mm) exit orifice is laser drilled in the semipermeable walled bi-layered tablet to provide contact of the 3-aminopropyl-n-butyl-phosphinic acid—lauryl sulfate complex layer with the exterior of the delivery device. The dosage form is dried to remove any residual solvent and water.

The in vitro dissolution rate of the dosage form is determined by placing a dosage form in the metal coil sample holders attached to a USP Type VII bath indexer in a constant temperature water bath at 37° C. Aliquots of the release media are injected into a chromatographic system to quantify the amounts of drug released into a medium simulating artificial gastric fluid (AGF) during each testing interval. The results are recorded.

### Example 4

Matrix Dosage Form

A matrix dosage form according to the present invention is prepared as follows. 200 grams of 3-aminopropyl-n-butyl-phosphinic acid—lauryl sulfate complex prepared as in Example 1, 25 grams of hydroxypropyl methylcellulose having a number average molecular weight of 9,200 grams per mole, and 15 grams of hydroxypropyl methylcellulose having a molecular weight of 242,000 grams per mole, are passed through a screen having a mesh size of 40 wires per inch. The celluloses each have an average hydroxyl content of 8 weight percent and an average methoxyl content of 22 weight percent. The resulting sized powders are tumble mixed. Anhydrous ethyl alcohol is added slowly to the mixed powders with stirring until a dough consistency is produced. The damp mass is then extruded through a 20 mesh screen and air dried overnight. The resulting dried material is re-screened through a 20 mesh screen to form the final granules. 2 grams of the tabletting lubricant, magnesium stearate, which are sized through an 80 mesh screen, are then tumbled into the granules.

752 mg of the resulting granulation is placed in a die cavity having an inside diameter of 9/32 inch and compressed with deep concave punch tooling using a pressure head of 2 tons. This forms a longitudinal capsule core having an overall length, including the rounded ends, of 0.691 inch. The cylindrical body of the capsule, from tablet land to tablet land, span a distance of 12 mm. Each core contains a unit dose of 3-aminopropyl-n-butyl-phosphinic acid—lauryl sulfate complex of 622 mg, (250 mg 3-aminopropyl-n-butyl-phosphinic acid equivalent).

### Example 5

Modified Matrix Dosage Form

A modified matrix dosage form according to the present invention is prepared as follows. A matrix delivery system according to Example 4 above is prepared to form a core. The core contains a unit dose of 3-aminopropyl-n-butyl-phosphinic acid—lauryl sulfate complex of 200 mg. Rings of polyethylene having an inside diameter of 9/32 inch, a wall thickness of 0.013 inch, and a width of 2 mm are then fabricated. These rings, or bands, are press fitted onto the core to complete the dosage form.

### Example 6

### Preparation of Two Drug Layer Osmotic Dosage Form Comprising a 3-Aminopropyl-n-Butyl-Phosphinic Acid—Lauryl Sulfate Complex

A dosage form comprising a layer of 3-aminopropyl-n-butyl-phosphinate chloride and a layer of 3-aminopropyl-n-butyl-phosphinic acid—lauryl sulfate complex is prepared as follows.

10 grams of 3-aminopropyl-n-butyl-phosphinate, 1.18 g of polyethylene oxide of 100,000 molecular weight, and 0.53 g of polyvinylpyrrolidone having molecular weight of about 38,000 are dry blended in a conventional blender for 20 minutes to yield a homogenous blend. Next, 4 mL denatured anhydrous alcohol is added slowly, with the mixer continuously blending, to the three component dry blend. The mixing is continued for another 5 to 8 minutes. The blended wet composition is passed through a 16 mesh screen and dried overnight at room temperature. Then, the dry granules are passed through a 16 mesh screen and 0.06 g of magnesium stearate are added and all the ingredients are dry blended for 5 minutes. The fresh granules are ready for formulation as the initial dosage layer in the dosage form. The granules are comprised of 85.0 wt % 3-aminopropyl-n-butyl-phosphinate hydrochloride, 10.0 wt % polyethylene oxide of 100,000 molecular weight, 4.5 wt % polyvinylpyrrolidone having a molecular weight of about 35,000 to 40,000, and 0.5 wt % magnesium stearate.

The 3-aminopropyl-n-butyl-phosphinic acid—lauryl sulfate complex layer in the dosage form is prepared as follows. First, 9.30 grams of 3-aminopropyl-n-butyl-phosphinic acid—lauryl sulfate complex, prepared as described in Example 1, 0.50 g polyethylene oxide of 5,000,000 molecular weight, 0.10 g of polyvinylpyrrolidone having molecular weight of about 38,000 are dry blended in a conventional blender for 20 minutes to yield a homogenous blend. Next, denatured anhydrous ethanol is added slowly to the blend with continuous mixing for 5 minutes. The blended wet composition is passed through a 16 mesh screen and dried overnight at room temperature. Then, the dry granules are passed through a 16 mesh screen and 0.10 g magnesium stearate are added and all the dry ingredients are dry blended for 5 minutes. The composition is comprised of 93.0 wt % 3-aminopropyl-n-butyl-phosphinic acid—lauryl sulfate complex, 5.0 wt % polyethylene oxide 5,000,000 molecular weight, 1.0 wt % polyvinylpyrrolidone having molecular weight of about 35,000 to 40,000 and 1.0 wt % magnesium stearate.

A push layer comprised of an osmopolymer hydrogel composition is prepared as follows. First, 58.67 g of pharmaceutically acceptable polyethylene oxide comprising a 7,000,000 molecular weight, 5 g Carbopol® 974P, 30 g sodium chloride and 1 g ferric oxide are separately screened through a 40 mesh screen. The screened ingredients are mixed with 5 g of hydroxypropylmethylcellulose of 9,200 molecular weight to produce a homogenous blend. Next, 50 mL of denatured anhydrous alcohol is added slowly to the blend with continuous mixing for 5 minutes. Then, 0.080 g of butylated hydroxytoluene is added followed by more blending. The freshly prepared granulation is passed through a 20 mesh screen and allowed to dry for 20 hours at room temperature (ambient). The dried ingredients are passed through a 20 mesh screen and 0.25 g of magnesium stearate was added and all the ingredients are blended for 5 minutes. The final composition is comprised of 58.7 wt % of polyethylene oxide, 30.0 wt % sodium chloride, 5.0 wt % Carbopol® 974P, 5.0 wt % hydroxypropylmethylcellulose, 1.0 wt % ferric oxide, 0.25 wt % magnesium stearate, and 0.08 wt % butylated hydroxytoluene.

The tri-layer dosage form is prepared as follows. First, 118 mg of the 3-aminopropyl-n-butyl-phosphinate composition is added to a punch and die set and tamped, then 535 mg of the 3-aminopropyl-n-butyl-phosphinic acid—lauryl sulfate complex composition is added to the die set as the second layer and again tamped. Then, 327 mg of the hydrogel composition is added and the three layers compressed under a compression force of 1.0 ton (1000 kg) into a 9/32 inch (0.714 cm) diameter punch die set, forming an intimate tri-layered core (tablet).

A semipermeable wall-forming composition is prepared comprising 80.0 wt % cellulose acetate having a 39.8% acetyl content and 20.0% polyoxyethylene-polyoxypropylene copolymer having a molecular weight of 7680-9510 by dissolving the ingredients in acetone in a 80:20 wt/wt composition to make a 5.0% solids solution. Placing the solution container in a warm water bath during this step accelerates the dissolution of the components. The wall-forming composition is sprayed onto and around the tri-layered core to provide a 60 to 80 mg thickness semi-permeable wall.

Next, a 40 mil (1.02 mm) exit orifice is laser drilled in the semipermeable walled tri-layered tablet to provide contact of the 3-aminopropyl-n-butyl-phosphinate chloride layer with the exterior of the delivery device. The dosage form is dried to remove any residual solvent and water.

The in vitro dissolution rates of the dosage form is determined by placing a dosage form in the metal coil sample holders attached to a USP Type VII bath indexer in a constant temperature water bath at 37° C. Aliquots of the release media are injected into a chromatographic system to quantify the amounts of drug released into a medium simulating artificial gastric fluid (AGF) during each testing interval. The average dissolution rates are determined.

### Example 7

### Gastric Retention System for Delivery of 3-Aminopropyl-n-Butyl-Phosphinic Acid—Lauryl Sulfate Complex

A dosage form according to the disclosure in U.S. Pat. No. 6,548,083 to Wong, et al., granted Apr. 15, 2003, entitled “Prolonged release active agent dosage form adapted for gastric retention”, and incorporated by reference herein in its entirety, is prepared with 3-aminopropyl-n-butyl-phosphinic acid—lauryl sulfate complex.

Eighteen grams of 3-aminopropyl-n-butyl-phosphinic acid—lauryl sulfate complex, prepared as in Example 1, and 3.6 grams of the gel-forming polymer polyethylene oxide, having a number average molecular weight of approximately 8 million grams per mole, are separately screened through a mesh having 40 wires per inch. The polyethylene oxide is supplied under the trade name Polyox.RTM. grade 308 as manufactured by Union Carbide Corporation, Danbury, Conn. The sized active agent and polymer are dry mixed. Then, 8.25 grams of a hydroattractant water-insoluble polymer, hydroxypropyl cellulose having a hydroxypropyl content of 10-13 weight percent and an average fiber particle size of 50 microns, is sieved through the 40-mesh screen and blended into the mixture. The hydroxypropyl cellulose is supplied as Low-Substituted Hydroxypropyl Cellulose grade 11 as manufactured by Shin-Etsu Chemical Company, Ltd., Tokyo, Japan. Anhydrous ethyl alcohol, specially denatured formula 3A, i.e., ethanol denatured with 5 volume percent methanol, is added to the mixture with stirring until a uniformly damp mass formed. This damp mass is extruded with pressure through a screen having 20 wires per inch. The extrudate is then allowed to air dry at room temperature overnight. After drying, the resulting extrudate is passed again through the 20-mesh sieve, forming granules. 0.15 Grams of the tableting lubricant, magnesium stearate, are passed through a sieve having 60 wires per inch. The sized 60-mesh lubricant is then tumbled into the granules to produce the finished granulation.

Portions of the resulting granulation are weighed and compacted with caplet-shaped tooling on a Carver press at pressure head of 1.5 tons. Each tablet weighs approximately 1042 mg and contains approximately 625 mg of the active agent. The shape of the tablet has approximately cylindrical proportions. The diameter is approximately 7.6 millimeters (mm) and the length was approximately 22 mm.

A tube of polyolefin material having an outside diameter of 7.7 mm and having a wall thickness of 0.25 mm is sliced with a razor to produce rings. The width of each ring is approximately 3 mm. One ring is then press fitted onto each caplet such that the ring, or band, is located approximately at the midpoint of the length of the caplet. This step completes the fabrication procedure of the 625 mg banded caplet.

1. A dosage form comprising:
(i) a controlled delivery dosing structure comprising structure that controllably delivers a drug;
(ii) the drug being selected from the group consisting of 3-aminopropyl-n-butyl-phosphinic acid; structural homologs thereof; complexes that comprise 3-aminopropyl-n-butyl-phosphinic acid or structural homologs thereof; pharmaceutically acceptable salts of 3-aminopropyl-n-butyl-phosphinic acid or structural homologs thereof; and mixtures of the above;
wherein at least a portion of the drug is contained by the controlled delivery dosing structure; and
wherein the controlled delivery dosing structure is adapted to controllably deliver the portion of the drug contained by the controlled delivery dosing structure at a rate that is effective to, after a single administration of the dosage form to a patient:
a. provide a Cmax ranging from about 0.01 to about 700 μmol/L,
b. provide an AUC from about 30 to about 1500 h·μmol/L, and
c. maintain a plasma drug concentration that is at least about fifteen percent of the Cmax throughout a window of at least about ten hours duration.

2. The dosage form of claim 1, wherein the window has a duration of at least about twelve hours.

3. The dosage form of claim 1, wherein the window has a duration of at least about sixteen hours.

4. The dosage form of claim 1, wherein the window has a duration of at least about eighteen hours.

5. The dosage form of claim 1, wherein the window has a duration of at least about twenty hours.

6. The dosage form of claim 1, wherein the Cmax ranges from about 10 to about 500 μmol/L.

7. The dosage form of claim 6, wherein the Cmax ranges from about 30 to about 300 μmol/L.

8. The dosage form of claim 1, wherein the AUC ranges from about 50 to about 1200 h·μmol/L.

9. The dosage form of claim 8, wherein the AUC ranges from about 10 to about 1000 h·μmol/L.

10. The dosage form of claim 1, wherein the complexes comprise a transport moiety that comprises alkyl sulfate salts.

11. The dosage form of claim 10, wherein the transport moiety comprises sodium lauryl sulfate.

12. The dosage form of claim 1, wherein the dosage form is a multiple unit dosage form.

13. A method comprising:
administering to a patient in need thereof a dosage form comprising a controlled delivery dosing structure comprising structure adapted to controllably deliver a drug, wherein the drug is selected from the group consisting of 3-aminopropyl-n-butyl-phosphinic acid, structural homologs thereof, complexes that comprise 3-aminopropyl-n-butyl-phosphinic acid or structural homologs thereof, pharmaceutically acceptable salts of 3-aminopropyl-n-butyl-phosphinic acid or structural homologs thereof, and mixtures of the above; and wherein at least a portion of the drug is contained by the controlled delivery dosing structure; and
controllably delivering the portion of the drug contained by the controlled delivery dosing structure at a rate that is effective to, after a single administration of the dosage form to a patient:
a. provide a Cmax ranging from about 0.01 to about 700 μmol/L,
b. provide an AUC (zero to infinity) from about 30 to about 1500 h·μmol/L, and
c. maintain a plasma drug concentration that is at least about fifteen percent of the Cmax throughout a window of at least about ten hours duration.

14. The method of claim 13, wherein the window has a duration of at least about twelve hours.

15. The method of claim 13, wherein the window has a duration of at least about sixteen hours.

16. The method of claim 13, wherein the window has a duration of at least about eighteen hours.

17. The method of claim 13, wherein the window has a duration of at least about twenty hours.

18. The method of claim 13, wherein the Cmax ranges from about 10 to about 500 μmol/L.

19. The method of claim 18, wherein the Cmax ranges from about 30 to about 300 μmol/L.

20. The method of claim 13, wherein the AUC ranges from about 50 to about 1200 h·μmol/L.

21. The method of claim 20, wherein the AUC ranges from about 10 to about 1000 h·μmol/L.

22. The method of claim 13, wherein the complexes comprise a transport moiety that comprises an alkyl sulfate salt.

23. The method of claim 22, wherein the transport moiety comprises sodium lauryl sulfate.

24. The method of claim 13, wherein the dosage form is a multiple unit dosage form.

25. A method comprising:
orally delivering a drug to a patient in need thereof at a substantially zero order delivery rate during a window;
wherein the drug is selected from the group consisting of structural homologs of 3-aminopropyl-n-butyl-phosphinic acid; complexes that comprise 3-aminopropyl-n-butyl-phosphinic acid or structural homologs thereof; pharmaceutically acceptable salts of structural homologs of 3-aminopropyl-n-butyl-phosphinic acid; and mixtures of the above; and
wherein the window has a duration of at least about ten hours.

26. The method of claim 25, wherein the drug is selected from the group consisting of complexes that comprise 3-aminopropyl-n-butyl-phosphinic acid or structural homologs thereof.

27. The method of claim 26, wherein the window has a duration of at least about twelve hours.

28. The method of claim 26, wherein the window has a duration of at least about sixteen hours.

29. The method of claim 25, wherein the window has a duration of at least about eighteen hours.

30. The method of claim 25, wherein the window has a duration of at least about twenty hours.

31. A dosage form comprising:
an oral controlled delivery dosing structure that is adapted to controllably deliver orally a drug at a substantially zero order delivery rate a during a window;
wherein the drug is selected from the group consisting of structural homologs of 3-aminopropyl-n-butyl-phosphinic acid; complexes that comprise 3-aminopropyl-n-butyl-phosphinic acid or structural homologs thereof; pharmaceutically acceptable salts of structural homologs of 3-aminopropyl-n-butyl-phosphinic acid; and mixtures of the above; and
wherein the window has a duration of at least about ten hours.

32. The dosage form of claim 31, wherein the drug is selected from the group consisting of complexes that comprise 3-aminopropyl-n-butyl-phosphinic acid or structural homologs thereof.

33. The dosage form of claim 31, wherein the window has a duration of at least about twelve hours following oral delivery.

34. The dosage form of claim 33, wherein the window has a duration of at least about sixteen hours following oral delivery.

35. The dosage form of claim 34, wherein the window has a duration of at least about eighteen hours following oral delivery.

36. The dosage form of claim 35, wherein the window has a duration of at least about twenty hours following oral delivery.

37. The dosage form of claim 31, wherein the dosage form is a multiple unit dosage form.

38. A dosage form comprising
(i) a controlled delivery dosing structure comprising structure that controllably delivers a drug;
(ii) the drug being selected from the group consisting of structural homologs of 3-aminopropyl-n-butyl-phosphinic acid; complexes that comprise 3-aminopropyl-n-butyl-phosphinic acid or structural homologs thereof; pharmaceutically acceptable salts of structural homologs of 3-aminopropyl-n-butyl-phosphinic acid; and mixtures of the above;
wherein at least a portion of the drug is contained by the controlled delivery dosing structure; and
wherein the controlled delivery dosing structure is adapted to controllably deliver the portion of the drug contained by the controlled delivery dosing structure in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 24 hrs.

39. A method of administering to a patient in need thereof a dose of a drug comprising:
administering the drug to a patient in a delivery dose pattern of from about 0 wt % to about 20 wt % in about 0 to about 4 hrs, about 20 wt % to about 50 wt % in about 0 to about 8 hrs, about 55 wt % to about 85 wt % in about 0 to about 14 hrs, and about 80 wt % to about 100 wt % in about 0 to about 24 hrs; and
wherein the drug is selected from the group consisting of structural homologs of 3-aminopropyl-n-butyl-phosphinic acid; complexes that comprise 3-aminopropyl-n-butyl-phosphinic acid or structural homologs thereof; pharmaceutically acceptable salts of structural homologs of 3-aminopropyl-n-butyl-phosphinic acid; and mixtures of the above.

40. A substance comprising:
a complex that comprises 3-aminopropyl-n-butyl-phosphinic acid or structural homologs thereof; and a transport moiety.

41. A composition comprising:
the substance of claim 40 and a pharmaceutically acceptable carrier or excipient.

42. The composition of claim 41, wherein the pharmaceutically acceptable carrier or excipient comprises an osmagent, a binder, or a lubricant.

43. The substance of claim 41, wherein the transport moiety comprises an alkyl sulfate salt.

44. The substance of claim 43, wherein the alkyl sulfate salt comprises a C6-C18 alkyl sulfate salt.

45. The substance of claim 44, wherein the C6-C18 alkyl sulfate salt comprises a C12 alkyl sulfate salt.

46. An oral dosage form comprising the composition of claim 41.

47. An oral dosage form, comprising a complex that comprises 3-aminopropyl-n-butyl-phosphinic acid or structural homologs thereof, and a C12 alkyl sulfate salt, which complex is present in an amount effective to antagonize gamma-aminopropylbutyric acid B receptors in a patient for a window having a duration of at least about ten hours.

48. The dosage form of claim 47, wherein the window has a duration of at least about 12 hours.

49. The dosage form of claim 48, wherein the window has a duration of at least about 16 hours.

50. The dosage form of claim 49, wherein the window has a duration of at least about 20 hours.

51. The dosage form of claim 50, wherein the window has a duration of at least about 24 hours.

52. The dosage form of claim 47, wherein the dosage form comprises a weight equivalent of 3-aminopropyl-n-butyl phosphinic acid ranging from about 100 mg to about 1500 mg.

53. The dosage form of claim 52, wherein the dosage form comprises a weight equivalent of 3-aminopropyl-n-butyl phosphinic acid ranging from about 300 mg to about 1200 mg.

54. The dosage form of claim 53, wherein the dosage form comprises a weight equivalent of 3-aminopropyl-n-butyl phosphinic acid ranging from about 400 mg to about 900 mg.

55. A dosage form comprising:
(i) a controlled delivery dosing structure comprising structure that controllably delivers a drug;
(ii) the drug comprising a complex that comprises 3-aminopropyl-n-butyl-phosphinic acid or structural homologs thereof, and a C12 alkyl sulfate salt;
wherein at least a portion of the drug is contained by the controlled delivery dosing structure; and
wherein the controlled delivery dosing structure controllably delivers the portion of the drug contained by the controlled delivery dosing structure at a rate that is effective to, after a single administration of the dosage form to a patient:
a. provide a Cmax ranging from about 0.01 to about 700 μmol/L,
b. provide an AUC (zero to infinity) from about 30 to about 1500 h·μmol/L, and
c. maintain a plasma drug concentration that is at least about fifteen percent of the Cmax throughout a window of at least about ten hours duration.

56. The dosage form of claim 55, wherein the window has a duration of at least about twelve hours.

57. The dosage form of claim 56, wherein the window has a duration of at least about sixteen hours.

58. The dosage form of claim 57, wherein the window has a duration of at least about eighteen hours.

59. The dosage form of claim 58, wherein the window has a duration of at least about twenty hours.

60. The dosage form of claim 55, wherein the dosage form comprises a weight equivalent of 3-aminopropyl-n-butyl phosphinic acid ranging from about 100 mg to about 1500 mg.

61. The dosage form of claim 60, wherein the dosage form comprises a weight equivalent of 3-aminopropyl-n-butyl phosphinic acid ranging from about 300 mg to about 1200 mg.

62. The dosage form of claim 61, wherein the dosage form comprises a weight equivalent of 3-aminopropyl-n-butyl phosphinic acid ranging from about 400 mg to about 900 mg.

63. The dosage form of claim 55, wherein the C12 alkyl sulfate salt comprises sodium lauryl sulfate.

64. The dosage form of claim 55, wherein the Cmax ranges from about 10 to about 500 μmol/L.

65. The dosage form of claim 64, wherein the Cmax ranges from about 30 to about 300 μmol/L.

66. The dosage form of claim 55, wherein the AUC ranges from about 50 to about 1200 h·μmol/L.

67. The dosage form of claim 66, wherein the AUC ranges from about 10 to about 1000 h·μmol/L.

68. The dosage form of claim 55, wherein the dosage form is a multiple unit dosage form.

69. A method of improving absorption of 3-aminopropyl-n-butyl-phosphinic acid comprising:
providing a complex of 3-aminopropyl-n-butyl-phosphinic acid and a transport moiety; and
administering the complex to a patient in need thereof.

70. The method of claim 69, wherein the transport moiety comprises an alkyl sulfate salt.

71. The method of claim 70, wherein the alkyl sulfate salt comprises a C6-C18 alkyl sulfate salt.

72. The method of claim 71, wherein the C6-C18 alkyl sulfate salt comprises a C12 alkyl sulfate salt.

73. The method of claim 69, wherein the complex is administered orally, and the improved absorption comprises improved oral absorption.

74. The method of claim 73, wherein the improved oral absorption comprises improved lower gastrointestinal tract absorption.

75. The method of claim 73, wherein the improved oral absorption comprises improved upper gastrointestinal tract absorption.
